(19) United States
(12) Patent Application Publication
(10) Pub. No.: US 2004/0115814 A1
(43) Pub. Date: Jun. 17, 2004

(54) Efficient generation of stable expression cell lines through the use of scorable homeostatic reporter genes

(75) Inventor: Robert B. DuBridge, Belmont, CA (US)

Correspondence Address: HOWREY SIMON ARNOLD & WHITE, LLP, BOX 34, 301 RAVENSWOOD AVE., MENLO PARK, CA, 94025, US

(73) Assignee: Protein Design Labs, Inc., Fremont, CA

(21) Appl. No.: 10/676,476

(22) Filed: Sep. 30, 2003

Related U.S. Application Data
(60) Provisional application No. 60/415,216, filed on Sep. 30, 2002.

(51) Int. Cl. C12Q 1/68; C12N 15/85

(52) U.S. Cl. 435/455; 435/320.1

(57) ABSTRACT

The present invention provides methods for site-specific recombination in a cell, as well as vectors which can be employed in such methods. The methods and vectors of the present invention can be used to obtain persistent gene expression in a cell and to modulate gene expression. One preferred method according to the invention comprises contacting a cell with a vector comprising an origin of replication functional in mammalian cells located between first and second recombining sites located in parallel. Another preferred method comprises, in part, contacting a cell with a vector comprising first and second recombining sites in antiparallel orientations such that the vector is internalized by the cell. In both methods, the cell is further provided with a site-specific recombinase that effects recombination between the first and second recombining sites of the vector.

### CROSS-REFERENCE TO RELATED APPLICATIONS

This application claims the benefit of U.S. Provisional Application No. 60/415,216, filed Sep. 30, 2002 under 35 U.S.C. § 119(e).

### FIELD OF THE INVENTION

This invention relates to molecular biological techniques and systems for producing stable genetic expression of one or more recombinant molecules. Particularly, compositions, systems and methods are disclosed for producing recombinant cells capable of stable, reproducible genetic expression.

### BACKGROUND OF THE INVENTION

Stable, high level expression systems are routinely produced by introducing recombinant genes to competent cells through insertion of the recombinant gene at random locations in the cellular genetic material by non-homologous recombination. (See, e.g., U.S. Pat. No. 5,202,238 and PCT/IB95 (00014)). This approach requires several rounds of selection and clonal expansion to produce an acceptable expression system. Moreover, this process must be repeated every time an expression system for a new gene is sought. To produce expression systems for multi-subunit complexes by this random process increases the complexity of acquiring the expression system by several orders of magnitude.

While this approach has proven successful, there are a number of problems with the system because of the random nature of the integration event. Some of these locations where recombinant genes are inserted are incapable of supporting transcriptional events at all. These problems exist because expression levels are greatly influenced by the effects of the local genetic environment at the gene locus, a phenomenon well documented in the literature and generally referred to as “position effects” (for example, see Al-Shawi et al, Mol. Cell. Biol., 10: 1192-1198 (1990); Yoshimura et al, Mol. Cell. Biol., 7:1296-1299 (1987)). As the vast majority of mammalian DNA is in a transcriptionally inactive state, random integration methods offer no control over the transcriptional fate of the integrated DNA. Consequently, wide variations in the expression level of integrated genes can occur, depending on the site of integration. For example, integration of exogenous DNA into inactive or transcriptionally “silent” regions of the genome will result in little or no expression. By contrast, integration into a transcriptionally active site may result in high expression.

Recombinase-mediated exchange has been described for homologous recombination of transgenes at defined sites in the genome. (See, e.g., U.S. Pat. Nos. 5,654,182, 5,677,177 and 5,885,836, incorporated herein in its entirety). Although recombinase-meditated systems allow the directed exchange of transgenes, achieving stable, high-efficient expressors of integrated transgenes is still cumbersome and requires large numbers of screened clones in order to select desirable integrated cells.

Therefore, when the goal of the work is to obtain a high level of gene expression, as is typically the desired outcome of genetic engineering production methods, it is generally necessary to screen large numbers of transfectants to find such a high producing clone. Additionally, random integration of exogenous DNA into the genome can in some instances disrupt important cellular genes, resulting in an altered phenotype. These factors can make the generation of high expressing stable mammalian cell lines a complicated, laborious and slow process.

### SUMMARY OF THE INVENTION

The invention provides systems and methods for detecting and utilizing recombinant expression constructs inserted into genomic loci that support advantageous levels of transcriptional activity, and provide for the production of well-characterized and reproducible expression systems. The result is a rapid and efficient means of producing and identifying high expression recombinant cell populations that universally exchange genetic segments for protein production or other molecular recombination uses. The reproducibility of the system also allows for accelerated production, characterization, and transfer of production cell lines into GMP manufacturing facilities.

In one embodiment, the invention comprises a universal site-specific expression system comprising an integration cassette. The integration cassette has a promoter operably linked to an exchangeable reporter segment having two recombinase recognition sites flanking a scorable homeostatic reporter element encoding at least one scorable reporter gene, which may also include at least one gene encoding an exchangeable reporter. Generally speaking, scorable homeostatic reporter elements and their products do not kill the cell, and the integration cassette or the target segment may optionally comprise the rec element(s). The integration cassette can be stably and randomly inserted at one or more discrete genomic positions in cells of a cell population.

The embodiment also comprises a target cassette, having a target segment comprising two recombinase recognition sites flanking a target element encoding a molecule of choice, which can be either a protein or a nucleic acid, or both. At least one rec element encoding a recombinase activity recognizing the recombinase recognition sites of the exchangeable reporter segment and the exchangeable target segment may also be included. In some aspects of the embodiment, the recombinase activity comprises two recombinase activities from the group Flp, Cre, Int, Sin or Hin.

The embodiment functions by the exchangeable reporter segment of the integration cassette being exchanged with the exchangeable target segment. This is accomplished by transforming cells comprising the integration cassette with a rec element and the exchangeable target segment, resulting in the site specific integration of the target into the site previously occupied by the exchangeable reporter segment. Multiple exchangeable target segments may be used with the same or different target sites having appropriate recombinase recognition sites.

An optional feature of the system is a TAG sequence included in the integration cassette that is linked in-frame to the first homeostatic reporter element. TAG sequences take a variety of forms including, but not limited to, binding molecules, epitope tags, fluorescent tags, enzymes, and the like.

The above embodied system can be further extended by inclusion of a second integration cassette structurally similar to the first integration cassette described above, but may comprise a separately scorable homeostatic reporter element. This second integration cassette is used to transform the recombinant cell population comprising the first integration cassette discussed in previous paragraphs, where it inserts itself stably and randomly at one or more discrete genomic positions, e.g., discrete from the insertion site(s) of the first integration cassette.

A second exchangeable target segment is also included in this extended embodiment, structurally similar to the first exchangeable target segment discussed above, but having a different target element sequence. In addition to recognizing the recombinase recognition sites of the first set of exchangeable segments, the recombinase activity may also recognize the recombinase recognition sites of the second set of exchangeable segments. This arrangement allows swapping of target segments with their respective reporter segments when they are present in the same cell, provided the recombinase activity is also present. Alternatively, a second recombinase activity may be introduced that recognizes only the recombinase recognition sites of the second set of exchangeable segments, and therefore allows independent exchange of the second exchangeable target segment from the first exchangeable target segment.

In some aspects, the first and second target elements each encode one subunit of a protein complex, which can be an antibody. In other aspects the first and second target elements are, or may include, polylinkers comprising one or more cloning sites. One or both of the integration cassettes can also comprise a TAG sequence linked in-frame to the respective homeostatic reporter element.

An antibody producing cell population is also contemplated in the invention. Each cell of this population comprises two integration cassettes supporting the same transcriptional rate. One integration cassette produces the heavy chain and the other produces the light chain. The cell population can be expanded from a single cell containing the pair of equipotent integration cassettes, or the population can comprise cells with their respective integration cassettes distributed in a heterogeneous manner. In the context of this embodiment, “antibody” refers to an antibody, or fragment thereof, e.g., capable of specifically binding an antigenic component.

The concept of antibody-producing cell lines can be extended to another embodiment of the invention; a plastic antibody library comprising a cell population where each cell of the cell population includes a pair of integration cassettes inserted into the cellular genome as described above. In the selection process, cells are isolated where the expression levels of both integration cassettes of the cell are at similar or the same level. As one integration cassette has a target element comprising a nucleotide encoding an antibody light chain and the other integration cassette has a target element comprising the coding sequence for the antibody heavy chain, having integration cassettes that express both proteins equally aids in ensuring that the antibody is constructed correctly. The recombinant cells containing the integration cassettes can be clonal or heterogeneous in origin, meaning that the integration cassettes can be inserted in the same two genetic loci in every cell or in different loci, respectively. Alternative library constructions include varying the sequence of the nucleic acid encoding the light chain while keeping the corresponding heavy chain sequence constant; varying the sequence of the nucleic acid encoding the heavy chain while keeping the corresponding light chain sequence constant; or varying the sequence of both nucleic acids in each cell. In the context of this invention, the term “antibody” includes Fab and Fab′ antibody fragments.

Some aspects of the plastic antibody library feature integration cassettes encoding chimeric antibody peptides that include a secretory signal segment. In other aspects, the antibodies encoded by the library are humanized antibodies. Other aspects of the library produce fusion molecules from integration cassettes encoding an antibody peptide chain linked in-frame to a TAG sequence, as described earlier for coding sequences generally.

The invention also includes methods for creating a universal site-specific expression cell population. The method comprises:

1. Obtaining an integration cassette having a promoter operably linked to an exchangeable reporter segment with a structure as described above;

2. Introducing the integration cassette into competent cells to create recombinant cells that have the integration cassette inserted randomly at one or more discrete genomic positions.

3. Scoring the level of expression of the homeostatic reporter element; and,

4. Selecting cells having a level of expression for the first scorable homeostatic reporter element that has been predetermined as satisfactory.

The scorable homeostatic reporter element can be a cell surface antigen, a fluorescent protein or other suitable scorable reporter protein. Alternatively, the scorable homeostatic reporter element can be evaluated based on its effect on cellular viability. Moreover, the homeostatic reporter may encode more than one protein, including a scorable reporter and an exchangeable reporter.

The method can be extended to include introducing to the cell population an exchangeable target segment and a rec element encoding recombinase activity recognizing the recombinase recognition sites of the exchangeable target segment and the exchangeable reporter segment, leading to substitution of the exchangeable reporter segment with the exchangeable target segment in the integration cassette. The recombinase activity could be Flp, Cre, Int, Sin, Hin, or a combination of any of the same. In some aspects of the invention the rec element and the target segment comprise portions of the same vector.

Some aspects have the integration cassette inserted in nuclear chromosomes. In other aspects, the integration cassette(s) are inserted into extrachromosomal material, which can be endogenous or exogenous in origin. Still other aspects of the method include a scorable homeostatic reporter element encoding an antigen specifically recognized by an antibody coupled to a selectable marker. Binding of the antibody to the antigen indicates the expression level of the reporter. Other types of scorable homeostatic reporter elements are also envisioned. For example, the scorable homeostatic reporter element can encode a fluorescent protein and the scoring entail sorting the cells using a cell sorting technique, e.g., based on a fluorescent property of the fluorescent protein. The exchangeable reporter gene may or may not include a scoring capability, as with the scorable reporter gene. However, at least one of the genes encoded by the first scorable homeostatic reporter element should be scorable through any of the means disclosed herein. Exemplary target elements include nucleotides encoding hormones, interferons, cytokines, protease inhibitors, antisense RNAs, snRNAs and viral antigens. In some aspects of the method, these target elements are linked to a secretory signal segment.

To increase cell number, the method can be modified to include clonal expansion of a cell scoring at a predetermined level of expression for the scorable homeostatic reporter element. By clonal expansion, a single cell scoring at the predetermined level of expression for the scorable homeostatic reporter element is selected from a heterogenous transformed cell population. The single cell is propagated until a clonal population is established from which to perform transgene exchange.

Another way of extending the method is by adding the step of obtaining a second integration cassette constructed in an analogous manner to the first, which may have a different scorable homeostatic reporter element, and introducing this second integration cassette into recombinant cells having the first integration cassette. The cells are then scored and those identified as scoring a satisfactory level of expression of the second scorable homeostatic reporter element at a predetermined level of expression are selected to obtain a cell population having two discrete integration cassettes stably inserted within. A variant to this approach is to use the same scorable homeostatic reporter element in each integration cassette, but exchange the initial reporter out by recombining the first integration cassette with a target segment prior to introduction of the second integration cassette. When creating dual integration cassette transformants by this method, the target segments and rec elements used to transform the cell can all be on the same vector, different vectors, or introduced via two or more vectors. Some aspects of the invention utilize target elements encoding subunits of a multi-subunit complex. One or more of these subunits can be expressed from an integration cassette comprising a TAG sequence, creating a fusion protein consisting of the subunit fused to the product encoded by the TAG sequence. Still other aspects select cells where both integration cassettes express their target elements at the same level, a desirable feature particularly when the recombinant cells are engineered to produce antibodies. Alternatively, cells may be selected to produce the target elements at preselected ratios, e.g., where there is a ratio of subunits 1:2, 1:3, 2:3, 1:5, 1:10 or any desirable ratio that assists in the formation of a multi-subunit complex.

The invention also provides a universal site-specific expression cell population having an integration cassette comprising a scorable homeostatic reporter element stably and randomly inserted at one or more discrete genomic positions within each cell of the cell population, where the scorable homeostatic reporter element is expressed. The integration cassettes of this cell population can optionally comprise a TAG sequence linked in-frame to the homeostatic reporter element.

Still other embodiments of the invention include clonal universal site-specific expression cell lines where the integration cassette is stably inserted at the same discrete genetic position in each cell of the cell line.

The invention also includes a production cell line comprising an integration cassette. The integration cassette in one aspect of the embodiment is the same as that described above for the universal site-specific expression system, but has a target element encoding a protein of interest replacing the scorable reporter element. In one aspect, the first and second recombinase recognition sites are recognized by the same recombinase activity, while in other aspects the recognition sites are recognized by different recombinases. Regardless of which aspect is used, the recombinase(s) may be any recombinase mentioned herein or an equivalent thereof. Some aspects of the embodiment further comprise a TAG sequence, as described previously.

In addition to having the integration cassette integrated at a single genomic site, the invention includes having multiple integration cassettes integrated at multiple discrete genomic sites in the same cell. This aspect of the invention enhances the level of production of the protein(s) encoded by the target element. Typically, the target element in this aspect will encode the same protein(s) in each integration cassette, but may also comprise different proteins in each integration cassette at each multiple discrete genomic sites in the cell.

Other embodiments for enhancing production of proteins of interest is to include more than one transcriptional unit or nucleotide coding sequence in the target segment. These embodiments enhance production of the protein(s) of interest by including multiple copies of the coding sequence for the protein(s) in a single integration cassette.

### BRIEF DESCRIPTION OF THE DRAWINGS

FIG. 1a depicts an integration cassette comprising two transcriptional units, one driving the expression of an exchangeable reporter segment from an EF-1 a promoter, and the other expressing a blasticidin resistance gene.

FIG. 1b depicts two possible constructs for a vector comprising an exchangeable target segment. In this depiction, one of the vector constructs comprises an exchangeable target segment and a transcriptional unit for the expression of Flp recombinase. The other vector construct comprises only the exchangeable target segment.

FIG. 1c depicts a separate recombinase expression vector, which must be co-transfected with the vector containing an exchangeable target segment when no other source of a suitable recombinase activity is present in the system.

FIG. 2 is a cartoon illustrating random integration of integration cassettes into a cell. Briefly, competent cells are transformed with vectors comprising the integration cassette. Once within the cells, the integration cassette inserts itself at a random (or pseudo-random) position in the cellular genome. The cells then undergo selection for transformation and optimal features (e.g., quantity) of expression of the scorable homeostatic reporter element of the invention.

FIG. 3 is a diagrammatic example of a recombinase-catalyzed homologous recombination event between the pCE 1.0 CJA8 integration cassette and the CE 2.0BFH8 target segment described in examples 1 and 2. The figure shows the scorable homeostatic reporter element of the integration cassette being swapped with the target element of the target segment when the reporter and target segments are exchanged.

FIG. 4 is a schematic representation of the steps in constructing a cell line having dual integration cassettes.

FIG. 5 depicts target segment exchange with a reporter segment in the construction of an antibody-producing recombinant cell line. In this depiction the recombinase and both target segments are introduced to the cell via a common vector.

FIG. 6 depicts target segment exchange with a reporter segment in the construction of an antibody-producing recombinant cell line. In this depiction the recombinase and one of the target segments is introduced on one vector, the second target segment is introduced as part of a different vector.

FIG. 7 depicts target segment exchange with a reporter segment in the construction of an antibody-producing recombinant cell line. In this depiction the recombinase and the target segments are each introduced on separate vectors.

FIG. 8a depicts an exemplary integration cassette and exchangeable target segment vector for the production of an integration cassette construct expressing an antibody heavy chain.

FIG. 8b depicts an exemplary integration cassette and exchangeable target segment vector for the production of an integration cassette construct expressing an antibody light chain.

FIG. 9 depicts integration and exchangeable target cassettes CE 1.0-4.0 for the construction of an antibody library expression cell line containing cells expressing both heavy and light chain antibody subunits.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Antibody” or “Functional antibody” refers to a polypeptide ligand substantially encoded by an immunoglobulin gene or immunoglobulin genes, or fragments thereof, which specifically bind and recognize an epitope (e.g., an antigen). Antibodies are structurally defined by the interaction of two forms of polypeptide, one termed an “antibody light chain” and the other termed an “antibody heavy chain”. Each antibody light chain is covalently bound to an antibody heavy chain through one or more covalent bonds termed disulfide bridges. Each disulfide bridge consists of a disulfide bond between the γ-sulphide groups of two cystiene residues, one cysteine being part of the antibody heavy chain and the other cysteine being part of the antibody heavy chain. In addition to the covalent association with an antibody light chain, each antibody heavy chain can also be covalently associated with one or more antibody heavy chains. As with the association with antibody heavy and light chains, the interaction between two antibody heavy chains is through one or more disulphide bridges.

Generally, each antibody light chain and each antibody heavy chain is encoded in a separate transcriptional unit, or gene. The present invention however also envisions chimeric antibody genes encoding both heavy and light chains, including, but not limited to, chimeric genes where the coding sequences for heavy and light chains, two heavy chains, or a plurality of any combination of antibody heavy and light chains are joined by a nucleic acid encoding a linker peptide in-frame with the respective antibody-encoding sequences.

The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments discussed below.

The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

Antibodies can exist as intact immunoglobulins or as a number of well-characterized fragments produced by digestion with various peptidases. Thus, e.g., pepsin digests an antibody below the disulfide linkages in the hinge region to produce F(ab)′₂, a dimer of Fab which itself is a light chain joined to a truncated heavy chain by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see Fundamental Immunology (Paul ed., 3d ed. 1993)). While various antibody fragments are defined in terms of the digestion of an intact antibody, such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

Generally, a functional antibody is capable of specifically or selectively recognizing one or more epitopes found on an antigen. For example, an “antibody that specifically recognizes a product of the scorable homeostatic reporter element” is an antibody that under designated immunoassay conditions, binds to a protein encoded by a scorable homeostatic reporter element of the present invention with at least two times the background and does not substantially bind in a significant amount to other proteins that might be present in the sample. Typically a functional antibody will bind its antigen in a specific or selective reaction producing a signal at least twice that of the background signal or noise and more typically more than 10 to 100 times background, in a manner that is determinative of the presence of the antigen in a heterogeneous population of antigens and other biologics.

For preparation of monoclonal or polyclonal antibodies, many techniques can be used. See, e.g., Kohler & Milstein, Nature 256:495497 (1975); Kozbor et al., Immunology Today 4: 72 (1983); Cole et al., pp. 77-96 in Monoclonal Antibodies and Cancer Therapy, Alan R. Liss, Inc. (1985). Techniques for the production of single chain antibodies (U.S. Pat. No. 4,946,778) can also be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

“Cell population” as used herein means a collection of cells. A “clonal cell population” is one where each cell of the population originates from the same precursor cell, and thus are essentially genetically identical.

A “heterogeneous cell population” may refer to a collection of cells which belong to the same cell line or source (e.g., are related) but which differ in some material aspect, e.g., their phenotypic or genotypic makeup varies, or each cell of the population has integrated the same recombinant nucleic acid, but in a different genetic location (e.g., in a different chromosomal or plasmid location). As a consequence individuals within a heterogeneous cell population may not express the same proteins or exhibit the same biological activity.

A “recombinant cell population” is a cell population where each individual of the population has within its genetic makeup a nucleic acid sequence from an exogenous source. Recombinant cell populations can be clonal or heterogeneous and can be prokaryotic or eukaryotic in nature.

“Antigen” refers to substances which are capable, under appropriate conditions, of inducing a specific immune response and of reacting with the products of that response, e.g., with specific antibodies or specifically sensitized T-lymphocytes, or both. Antigens may be soluble substances, such as toxins and foreign proteins, or particulates, such as bacteria and tissue cells; however, only the portion of the protein or polysaccharide molecule known as the antigenic determinant (epitopes) combines with antibody or a specific receptor on a lymphocyte.

A “cell surface antigen” is a cell-associated component that can behave as an antigen without disrupting the integrity of the membrane of the cell expressing the antigen.

“Chromosomal” refers to both genetic (i.e. nucleic acid) and structural components of a cell associated with the native cellular chromosomes located e.g., in the cell nucleus, mitochondria or chloroplasts. “Extrachromosomal” refers to additional genetic material that is not chromosomal. Examples of extrachromosomal material includes plasmids and other nucleic acid based vectors that do not integrate into the native cellular chromosomes.

“Coupled to a selectable marker” refers to a trait that is associated with a gene that encodes a detectable activity, e.g., confers the ability to grow in medium lacking what would otherwise be an essential nutrient; in addition, a selectable marker may confer upon the cell in which the selectable marker is expressed, resistance to an antibiotic or drug. A selectable marker may be used to confer a particular phenotype upon a host cell. When a host cell must express a selectable marker to grow in selective medium, the marker is said to be a positive selectable marker (e.g., antibiotic resistance genes which confer the ability to grow in the presence of the appropriate antibiotic). See Eglitis (1991) Hum. Gene Therapy 2:195-201; Colbere-Garapin et al. (1982) Curr. Top. Microbiol. Imunol. 96:145-57. Selectable markers can also be used to select against host cells containing a particular gene; selectable markers used in this manner are referred to as negative selectable markers.

“Scorable homeostatic reporter element” refers to both genetic traits and the genes, typically recombinant in nature, that encode traits whose presence can be physically or chemically detected and quantified without adversely affecting the viability of the cell expressing the homeostatic reporter element. For example, the activity of an expressed enzyme can be scored by assaying for the enzyme activity. An example of a physically detectable trait is the fluorescence produced by green fluorescent proteins, which again can be measured and quantified, giving a determination of the amount of the fluorescent protein present, and hence expressed. This measurement and quantification of the expressed trait is termed “scoring the level of expression.”

When the level of expression of two scorable homeostatic reporter elements is equivalent, it is said that “the first level of expression is the same as the second level of expression.” “Equivalent expression” of two expression systems refers to levels of expression that do not differ by more than 2-fold from each other in terms of molar protein production, more preferably do not differ by more than 1.5-fold; and most preferably do not differ by more than 1.2-fold.

A preferred aspect of the scorable homeostatic reporters of the present invention is that they be scorable by a process that does not compromise the “viability” of the cell(s) expressing the reporter. Viability refers to the cells ability to carry out basic metabolic functions required to sustain life, including reproduction.

A “predetermined level of expression” is an expression level, typically a range of expression levels that are determined prior to expression analysis and used to make selections and generally considered when making future determinations.

“Discrete genomic position” or “discrete genomic position of insertion” in the context of this invention, refers to a genetic location occupied by a recombinant nucleic acid that is distinct and separate from genetic locations occupied by other recombinant nucleic acids. Two discrete genomic positions may be close together, but they should not overlap.

“Fluorescent protein” refers to a class of proteins comprising a fluorescent chromophore, the chromophore being formed from at least 3 amino acids and characterized by a cyclization reaction creating a p-hydroxybenzylidene-imidazolidinone chromophore. The chromophore does not contain a prosthetic group and is capable of emitting light of selective energy, the energy having been stored in the chromophore by previous illumination from an outside light source comprising the correct wavelength(s). Spontaneously fluorescent proteins can be of any structure, with a chromophore comprising any number of amino acids, provided that the chromophore comprises the p-hydroxybenzylidene-imidazolidinone ring structure, as detailed above. SFP's typically, but not exclusively, comprise a β-barrel structure such as that found in green fluorescent proteins and described in Chalfie et al., Science, 263, 802-805 (1994).

Fluorescent proteins characteristically exhibit “fluorescent properties,” which are the ability to produce, in response to an incident light of a particular wavelength absorbed by the protein, a light of longer wavelength.

“Nucleic acid” refers to a deoxyribonucleotide or ribonucleotide polymer in either single- or double-stranded form, and unless otherwise limited, encompasses known analogues of natural nucleotides that hybridize to nucleic acids in manner similar to naturally occurring nucleotides. Unless otherwise indicated, a particular nucleic acid sequence also describes the complementary sequence thereof.

“Nucleotide sequence” or “nucleic acid sequence” refers to the order placement of nucleotide bases in relation to each other as they appear in a polynucleotide.

A “non-human nucleotide sequence” is a nucleotide sequence that is not human in origin, including nucleotide sequences altered to reflect sequence characteristics found in human nucleotide sequences, provided the alteration is not complete (i.e., alteration to the point where the sequence is identical to one shown to exist in a human being).

Alterations of non-human sequences to give them human characteristics is termed “humanizing” and the resulting sequence termed a “humanized sequence.” See U.S. Pat. Nos. 6,407,213; 6,180,377; 5,530,101. Both nucleic acids and proteins can have humanized sequence alterations, typically to aid transcriptional and/or translational efficiency and avoid immune responses, respectively.

“Plastic antibody library” refers to a cell population capable of expressing a range of antibody species. Plastic antibody libraries differ from typical expression libraries in that the coding region for each antibody polypeptide can be swapped, as desired, for a different antibody polypeptide, producing a library that produces a different antibody repertoire from that produced by the original library. By limiting the swapping process to the coding region of the expression systems of the library, new libraries produced from old libraries are capable of producing a new antibody repertoire at the same expression levels as the previous antibody repertoire.

“Polycistronic element” refers to a nucleic acid encoding more than one protein. When a polycistronic element includes separate regulatory elements for two or more coding sequences, the combination of the regulatory elements and the coding sequence is termed a “transcriptional unit.”

A “promoter” is a DNA regulatory element capable of binding RNA polymerase in a cell and initiating transcription of a downstream (3′ direction) coding sequence. For purposes of defining the present invention, the promoter sequence includes, at its 3′ terminus, the transcription initiation site and extends upstream (in the 5′ direction) to include the minimum number of bases or elements necessary to initiate transcription at levels detectable above background. Within the promoter sequence will be found a transcription initiation site, as well as protein binding domains (consensus sequences) responsible for the binding of RNA polymerase. Eukaryotic promoters often, but not always, contain “TATA” boxes and “CAT” boxes.

Promoters (and other genetic regulatory elements) are typically “operably linked” to coding sequences. The term “operably linked” refers to a linkage of polynucleotide elements in a functional relationship. With regard to the present invention, the term “operably linked” refers to a functional linkage between a nucleic acid expression control sequence (such as a promoter, or an array of transcription factor binding sites) and a second nucleic acid sequence, e.g., wherein the expression control sequence directs transcription of the nucleic acid corresponding to the second sequence. Thus, a nucleic acid is “operably linked” when it is placed into a functional relationship with another nucleic acid sequence. Coding sequences of the present invention that are operably linked to promoters include selectable markers, scorable homeostatic reporter elements, exchangeable reporter segments and the like.

An “exchangeable target segment” is similar in construction to an exchangeable reporter segment. The two constructs differ in that the exchangeable target segment has a coding sequence for at least one desired expression product (the “target element”) located between the two recombinase recognition sites, instead of a scorable homeostatic reporter element. In some cases the exchangeable target segment will contain the coding sequence for a desired product and a coding sequence for a scorable or selectable marker. The segment can be constructed so that the translated product is a chimera, with the desirable expression product and the marker covalently linked through a peptide bond, or so that the desired expression product and the marker are translated into separate proteins.

In addition, the target element may also be expressed as a chimera containing a “secretory signal element.” A secretory signal element is a peptide sequence that directs the cellular machinery to export proteins containing the signal element. Thus a protein possessing a secretory signal element will be transported outside the cell.

An “integration cassette” of the present invention is a genetic construct having an exchangeable reporter segment operably linked to a promoter. The integration cassette is preferably designed to ease introduction into a cell, as the primary purpose of the integration cassette is to randomly integrate the construct into the genome of the cell, or otherwise create a situation where the integration cassette is stably transmitted to progeny of the initially transfected cell; e.g., the integration cassette is “stably inserted” into the genome of the cell. To this end, integration cassettes also include replicative and/or segregative episomes, e.g., artificial chromosomes and some high-copy number plasmids. Integration cassettes may also include selectable and/or scorable markers, as described below. Within the context of the present invention however, stable insertion does not preclude genetic exchanges between the exchange segments of the present invention catalyzed by rec element-encoded recombinase(s).

A “target cassette”, “target expression cassette” or “exchangeable target cassette” is an expression vector that can comprise target segments and optional rec elements in many combinations. Target cassettes generally allow for the introduction of target segments into cells and/or present the recombinase activity that allows for the exchange of genetic elements between compatible segments of the invention as disclosed herein. (For example, between an exchangeable reporter segment and an exchangeable target segment).

A “rec element” is a genetic construct capable of expressing one or more recombinases. To this end, a rec element contains regulatory sequences necessary to drive transcription of the recombinase coding sequence(s). These regulatory sequences typically include promoters and 3′ termination sequences. Generally rec promoters are constitutive promoters, but they need not be. In some embodiments, the promoter found in the rec element is constitutive. Other embodiments incorporate rec element promoters that are tissue or developmentally regulated.

“Recombinase” and “site-specific recombinase” refer to enzymes that catalyze a site-specific recombination event between two nucleic acid sequences. These enzymes include recombinases, transposases and integrases. The site where this recombination event occurs is termed a “recombinase recognition site” and is comprised of inverted palindromes separated by an asymmetric sequence. Examples of recombinase recognition sites include, but are not limited to, lox sites, att sites, dif sites and frt sites. For reviews of recombinases, see Sauer (1994) Current Opinion in Biotechnology, 5:521-527; Landy, Current Opinion in Biotechnology 3:699-707 (1993); and Sadowski (1993) FASEB 7:760-767.

The term “frt site” as used herein refers to a recombinase recognition site at which the product of the FLP gene of the yeast 2 micron plasmid, Flp recombinase, can catalyze site-specific recombination. Although the invention is not limited to the frt/Flp recombination system, the frt/Flp system is a preferred embodiment and is referred to repeatedly in the present application as one exemplary system.

“Recombinase activity” refers to the enzyme catalyzed exchange, insertion, or deletion of genetic material between two nucleic acid sequences through a recombination event occurring at or near sequence motifs present in the two sequences and recognized by the recombinase enzyme.

These sequence motifs recognized by the recombinase enzyme are termed “recombinase recognition sites.” Recombinase recognition sites are short nucleotide sequences and become the crossover regions during the site-specific recombination event. Examples of sequence-specific recombinase target sites include, but are not limited to, lox sites, att sites, dif sites and frt sites. Recombinase recognition sites are typically specific for a given recombinase though a particular recombinase may recognize different sites, and a single recombinase may mediate two different site-specific events.

Recombinases and recombinase recognition sites therefore allow for site-specific insertion, deletion of substitution of one nucleic acid with another. The present invention uses these site-specific manipulation tools to exchange coding regions within an expression system integrated into a cells DNA in a site-specific manner. Site-specific substitution of one coding sequence for another within a known, integrated expression construct is termed “site-specific expression,”and cells containing such integrated constructs are termed “site-specific expression cell lines.” The entire apparatus for conducting site-specific substitution of coding regions within a cell is termed a “site-specific expression system.”

“Restriction sites” are also short, enzyme-recognized sequence motifs found within a nucleic acid, but in the case of restriction sites, the motif is specifically recognized by an endonuclease activity, which cleaves a bond between two of the residues making up the restriction site. In the case of endonucleases recognizing restriction sites in duplexed DNA, a bond in each strand within the restriction site may be cleaved.

A protein is a molecule comprising predominantly amino acid residues linked through peptide bonds. Proteins generally consist of at least 20 amino acids, but can be extremely large, with a peptide backbone stretching over hundreds of amino acid residues.

Proteins can form complexes with other molecules, including other proteins, through covalent and/or non-covalent interactions. Predictably, such complexes are termed “protein complexes. When one or more of the molecules making up the complex are bound together by non-covalent forces, the complex is termed a “multi-subunit complex,” and the molecules being held together are referred to as “subunits.”

### DETAILED DESCRIPTION OF THE INVENTION

I. Introduction

The present invention provides compositions, systems and methods for identifying and utilizing advantageous genomic sites for expression of recombinant proteins. This is accomplished by randomly inserting plastic expression systems that permit exchange of their coding regions while leaving the remainder of the expression system, including the promoter, in place.

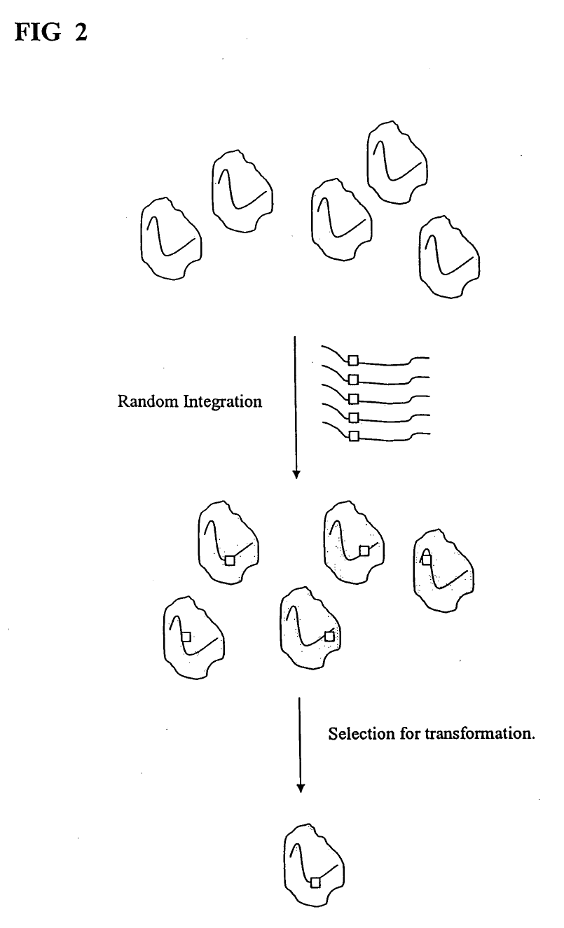

More specifically, the invention described herein provides integration cassettes that are inserted into cellular genetic material by a non-homologous recombination event. These integration cassettes comprise expression systems for selectable and scorable reporter genes that allow cells successfully transformed with the integration cassettes to be identified and the level of expression supported by the cassette at its site of insertion to be established. By monitoring the level of expression supported by a population of cells transformed with integration cassettes inserted at different genetic loci, cell populations supporting optimal expression features can be established. This approach is advantageous as it eliminates the need for repetitive rounds of selection and clonal expansion when a new gene is to be cloned. Instead, a prescreened cellular expression system of the present invention can be selected, and the gene of interest universally swapped into the system. This places the gene of interest under the control of a known promoter located at a reproducible site within the genome, e.g., characterized to support a given level of genetic expression. Moreover, as the expression systems of the present invention are stable and reusable, the locus of each integration cassette, particularly its genetic environment, can be characterized and understood in much greater detail than would be practical for the one-time “shotgun” approaches to cloning common in the field. A summary of the approach to constructing expression systems of the present invention is depicted diagrammatically in FIG. 2. This reproductability provides great advantages in a regulatory environment as the characteristics of production cell lines can be more reliably characterized and controlled.

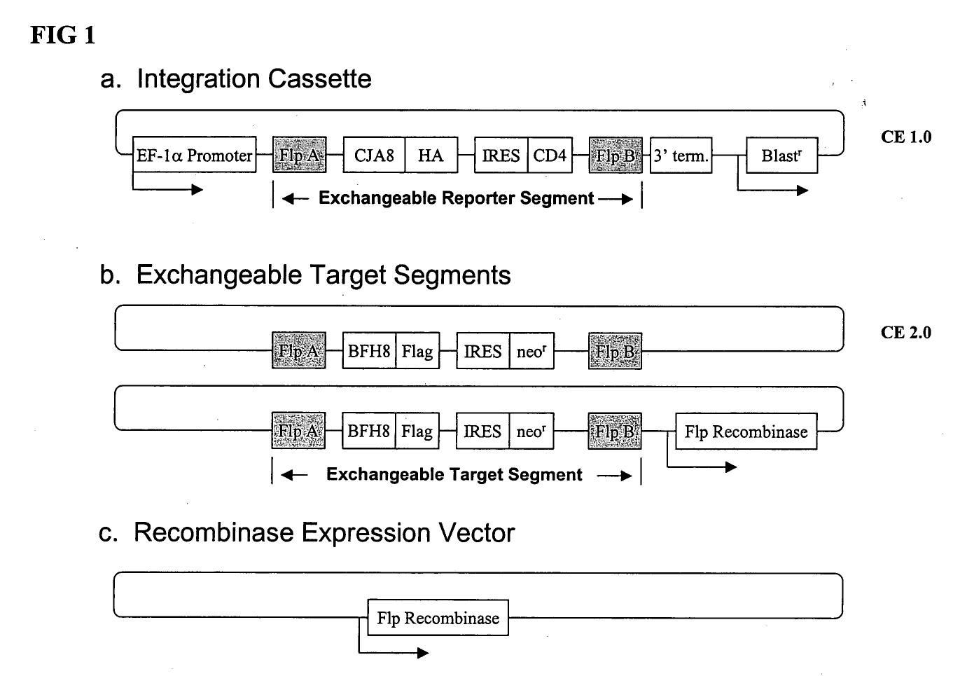

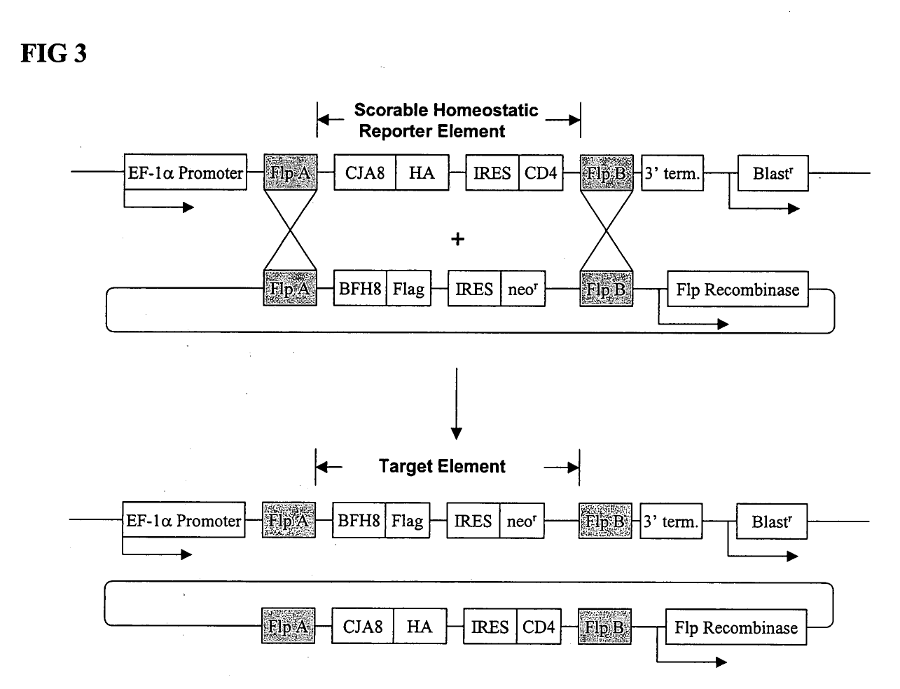

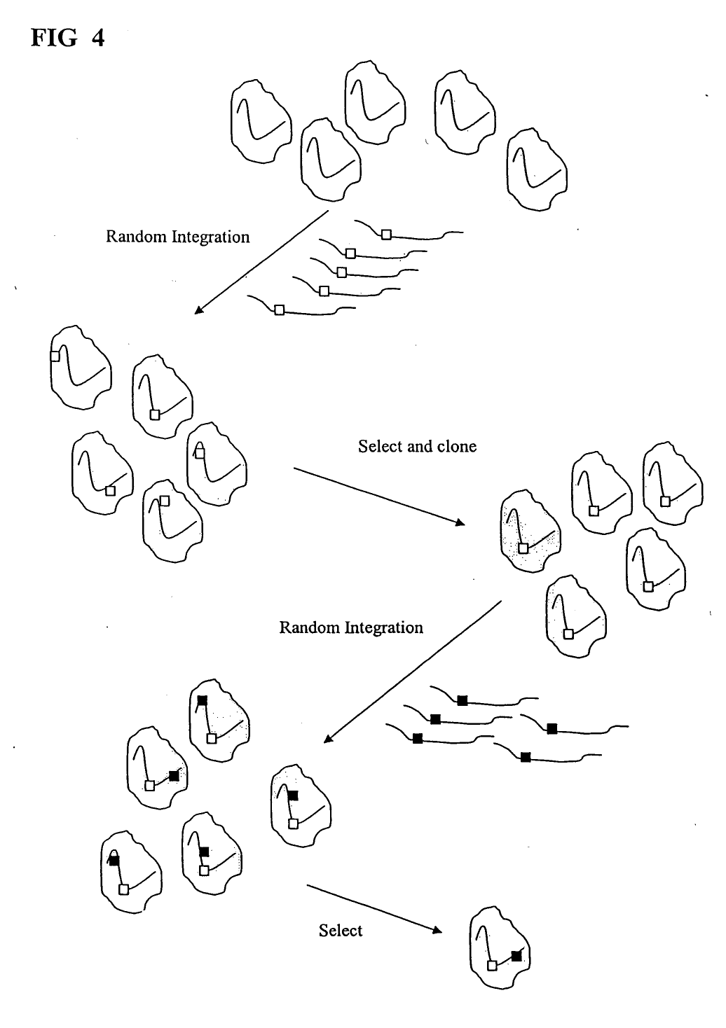

Swapping a gene of interest into a predetermined position of the genome is accomplished by the present invention through homologous recombination between recombinase recognition sequences. Recombinase recognition sequences are located in both the integration cassette inserted at the predetermined genomic position, and on a target segment comprising the gene of interest. The recombinase recognition sequences flank the coding regions that are to be swapped (see FIG. 1a.). Addition of a compatible corresponding recombinase activity to a system containing at least one compatible integration cassette and target segment catalyzes the “swapping” of coding sequences between the integration cassette and the target segment (see e.g., FIG. 3).

Because recombinase recognition sites of the present invention flank coding sequences, it is important that they do not contain interfering sequences, e.g., stop codons, or other genetic elements that would frustrate expression of the coding sequence between them. Consequently, the present invention includes methods for engineering recombinase recognition sites to minimize their impact on expression of the coding sequence(s) they flank.

Taking advantage of the stable constructs of the present invention, expression libraries are also included. Expression libraries of the present invention are particularly advantageous as, in addition to stability, the expression systems produced allow each member of the library to be expressed in a predictable manner at an identical genomic locus. This greatly simplifies evaluative screening as each library member is expressed in the context of a reproducible genetic environment equivalently; differences in response noted between library members can therefore be attributed to some effect outside transcriptional expression rates. As described herein, a variety of libraries can be constructed using cDNA's, genomic sequences, synthetic nucleic acids or combinations or derivatives of the same. In addition to providing recombinant proteins, these libraries can be used to study protein/protein interactions, as well as form therapeutics and other molecular reagents.

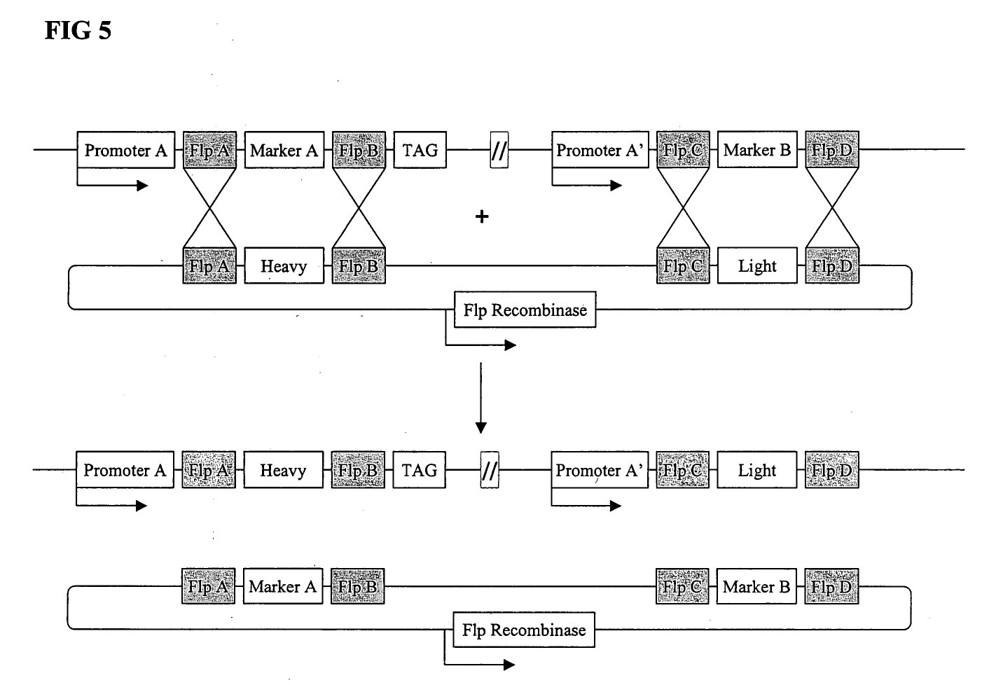

A particularly preferred feature of the present invention is the ability to create libraries whose members comprise more than one integration cassette-based expression construct. FIG. 4 illustrates the steps in constructing such a library. Briefly, a competent cell line/type is transformed with a first integration cassette. The transformed cell(s) having an integration cassette expressing at the desired level is selected and clonally expanded. These clones are then transformed with a second integration cassette and the selection process repeated for the second integration cassette. By using integration cassettes having different recombination recognition sequences, target segments can be constructed that specifically recombine with only one of the integration cassettes. This allows particular nucleic acids to be placed under the control of specific integration cassette promoters, giving complete control over the expression level of the nucleic acid. Using this system, expression libraries for multisubunit complexes can be made, such as the antibody-producing systems illustrated in FIGS. 5-7.

Another feature of the present invention is the use of TAG sequences, which allow proteins produced by the invention to be routinely tagged with scorable or selectable markers, or other fusion adducts, as an integral part of genetic expression. FIG. 5 illustrates the TAG sequence feature. A TAG sequence can encode a transcript to be linked to the coding sequence of the exchangeable segment. Exemplary TAG sequences that can act as scorable markers include epitope tags, binding tags such as hexahistidine (His-tag), poly lysine, receptors and antibodies, and fluorescent proteins. Although the TAG sequence is placed 3′ to the exchangeable segment in FIG. 5, orientations whereby the TAG sequence is 5′ to the exchangeable segment are also contemplated. Through the use of TAG sequences, dynamic studies of protein interaction can be performed. For example, a TAG sequence for a fluorescent protein can be included in the transcript of a protein of interest. A library of possible binding proteins for the protein of interest can then be TAGged with a second fluorescent protein suitable for FRET with the first fluorophore. By expressing the protein of interest with each of the library members, binding partners can be readily identified based on the fluorescent signal produced.

Again, by placing the TAG sequence outside the recombinase recognition site, libraries of fusion constructs can be formed whereby the product encoded by the TAG sequence is uniformly applied to the product of library members. For example, where the exchangeable segment comprises a diagnostic molecule, such as an enzyme for ELISA studies, the TAG sequence can encode a scorable marker.

The present invention also includes production cell lines for the producing biologics and enzymes. In the therapeutic arena, the production inputs and processes are highly regulated, and need to be carefully characterized and validated. A large component of the cost of biologic therapeutics is in the production and purification of the drug product, so high efficiency provides significant savings. The cost of commercial development includes a significant component of cost of capital, as the time throughout development before drug sales can be many years. Any means to shorten this time period can have dramatic impact on the cost of the drug to the patient.

II. Expression System Components

A. General Recombination Methods

Standard techniques for construction of the cassettes, segments, and corresponding vectors (recombinant elements) of the present invention are available. See (Sambrook, J., Fritsch, E. F., and Maniatis, T., Molecular Cloning, A Laboratory Manual 2nd ed. (1989); Kriegler, Gene Transfer and Expression: A Laboratory Manual (1990); and Current Protocols in Molecular Biology (Ausubel et al., eds., 1994). A variety of strategies are available for ligating fragments of DNA, the choice depending on the nature of the termini of the DNA fragments.

In preparing recombinant elements of the present invention, various DNA sequences may normally be inserted or substituted into a bacterial plasmid. Many convenient plasmids may be employed, which will be characterized by having a bacterial replication system, a marker which allows for selection in the bacterium and generally one or more unique, conveniently located restriction sites. These plasmids, referred to as vectors, may include such vectors as pACYC184, pACYC177, pBR322, pUC9, the particular plasmid being chosen based on the nature of the markers, the availability of convenient restriction sites, copy number, and the like. Thus, the sequence may be inserted into the vector at an appropriate restriction site(s), the resulting plasmid used to transform the E. coli host, the E. coli grown in an appropriate nutrient medium and the cells harvested and lysed and the plasmid recovered. One then defines a strategy that allows for the stepwise combination of the different fragments.

For nucleic acids, sizes are given in either kilobases (Kb) or base pairs (bp). These are typically estimates derived from agarose or acrylamide gel electrophoresis, from sequenced nucleic acids, or from published DNA sequences. Oligonucleotides that are not commercially available can be chemically synthesized, e.g., according to the solid phase phosphoramidite triester method first described by Beaucage & Caruthers, Tetrahedron Letts., 22:1859-1862 (1981), using an automated synthesizer, as described in Van Devanter et. al., Nucleic Acids Res., 12:6159-6168 (1984). Oligonucleotides are purified, e.g., by native acrylamide gel electrophoresis or by anion-exchange HPLC as described in Pearson & Reanier, J. Chrom., 255:137-149 (1983). Nucleic acid sequences may also be isolated and amplified using appropriate primers and PCR techniques, as described in e.g., Innis et al., PCR Protocols, A Guide to Methods and Applications, Academic Press, Inc. N.Y. (1990)).

Many ways of generating alterations in a given nucleic acid sequence are available. Such well-known methods include site-specific mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and others. See, e.g., Berger and Kimmel, Guide to Molecular Cloning Techniques, Methods in Enzymology, Volume 152 Academic Press, Inc., San Diego, Calif. (Berger); Sambrook et al., Molecular Cloning—A Laboratory Manual (2nd ed.) Vol. 1-3, Cold Spring Harbor Laboratory, Cold Spring Harbor Press, N.Y., (Sambrook) (1989); and Current Protocols in Molecular Biology, F. M. Ausubel et al, eds., Current Protocols, a joint venture between Greene Publishing Associates, Inc. and John Wiley & Sons, Inc., (1994 Supplement) (Ausubel); Pirrung et al., U.S. Pat. No. 5,143,854; and Fodor et al., Science, 251:767-77 (1991). Product information from manufacturers of biological reagents and experimental equipment also provide information useful in known biological methods. Such manufacturers include the SIGMA Chemical Company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), and Applied Biosystems (Foster City, Calif.), as well as many other commercial sources. Using these techniques, it is possible to insert or delete, at will, a polynucleotide into a DNA expression cassette described herein.

Site-directed mutagenesis techniques are described, for example, in Ling et al., “Approaches to DNA mutagenesis: an overview”, Anal Biochem., 254(2): 157-178 (1997); Dale et al., “In vitro mutagenesis”, Ann. Rev. Genet., 19:423-462 (1996); Botstein & Shortle, “Strategies and applications of in vitro mutagenesis”, Science, 229:1193-1201 (1985); Carter, “Site-directed mutagenesis”, Biochem. J, 237:1-7 (1986); and Kunkel, “The efficiency of oligonucleotide directed mutagenesis” in Nucleic Acids & Molecular Biology (Eckstein, F. and Lilley, D. M. J. eds., Springer Verlag, Berlin) (1987)); mutagenesis using uracil containing templates (Kunkel, “Rapid and efficient site-specific mutagenesis without phenotypic selection”, Proc. Natl. Acad. Sci. USA, 82:488-492 (1985); Kunkel et al., “Rapid and efficient site-specific mutagenesis without phenotypic selection”, Methods in Enzymol., 154:367-382 (1987); and Bass et al. (1988); oligonucleotide-directed mutagenesis (Methods in Enzymol., 100:468-500 (1983); Methods in Enzymol., 154:329-350 (1987); Zoller & Smith, “Oligonucleotide-directed mutagenesis using M 13-derived vectors: an efficient and general procedure for the production of point mutations in any DNA fragment”, Nucleic Acids Res., 10:6487-6500 (1982); Zoller & Smith “Oligonucleotide-directed mutagenesis of DNA fragments cloned into M13 vectors”, Methods in Enzymol., 100:468-500 (1983); and Zoller & Smith, “Oligonucleotide-directed mutagenesis: a simple method using two oligonucleotide primers and a single-stranded DNA template”, Methods in Enzymol., 154:329-350 (1987)); Taylor et al. (1985) “The rapid generation of oligonucleotide-directed mutations at high frequency using phosphorothioate-modified DNA”, Nucl. Acids Res., 13: 8765-8787 (1985); Nakamaye & Eckstein, “Inhibition of restriction endonuclease Nci I cleavage by phosphorothioate groups and its application to oligonucleotide-directed mutagenesis”, Nucl. Acids Res., 14:9679-9698 (1986); Sayers et al., “Y-T Exonucleases in phosphorothioate-based oligonucleotide-directed mutagenesis”, Nucl. Acids Res., 16:791-802 (1988); and Sayers et al. (1988); mutagenesis using gapped duplex DNA (Kramer et al., “The gapped duplex DNA approach to oligonucleotide-directed mutation construction”, Nucl. Acids Res., 12:9441-9456 (1984); Kramer & Fritz, “Oligonucleotide-directed construction of mutations via gapped duplex DNA”, Methods in Enzymol., 154:350-367 (1987); Kramer et al., “Improved enzymatic in vitro reactions in the gapped duplex DNA approach to oligonucleotide-directed construction of mutations”, Nucl. Acids Res., 16:7207 (1988); and Fritz et al., “Oligonucleotide-directed construction of mutations: a gapped duplex DNA procedure without enzymatic reactions in vitro”, Nucl. Acids Res., 16:6987-6999 (1988)).

Other techniques for altering DNA sequences include, for example; Wells et al., “Cassette mutagenesis: an efficient method for generation of multiple mutations at defined sites”, Gene, 34:315-323 (1985); and Grundstrom et al., “Oligonucleotide-directed mutagenesis by microscale ‘shot-gun’ gene synthesis”, Nucl. Acids Res., 13:3305-3316 (1985)), double-strand break repair (Mandecki, “Oligonucleotide-directed double-strand break repair in plasmids of Escherichia coli: a method for site-specific mutagenesis”, Proc. Natl. Acad. Sci. USA, 83:7177-7181 (1986); and Arnold, “Protein engineering for unusual environments”, Current Opinion in Biotechnology, 4:450-455 (1993)). Additional details on many of the above methods can be found in Methods in Enzymology Volume 154, which also describes useful controls for trouble-shooting problems with various mutagenesis methods.

The sequence of the isolated and synthetic oligonucleotides can be verified after cloning using, e.g., the chain termination method for sequencing double-stranded templates of Wallace et al., Gene, 16:21-26 (1981).

B. Suitable Vectors

In accordance with the invention, a vector may be used as a vehicle for delivering the integration cassettes, exchangeable target segments and recombinase expression systems of the present invention. In particular, vectors known in the art and those commercially available (and variants or derivatives thereof) may be engineered to include one or more recombination sites for use in the methods of the invention. Such vectors may be obtained from, for example, Vector Laboratories Inc., Invitrogen, Promega, Novagen, New England Biochemicals, Clontech, Boehringer Mannheim, Pharmacia, EpiCenter, OriGenes Technologies Inc., Stratagene, PerkinElmer, Pharmingen, Life Technologies, Inc., and Research Genetics. Such vectors may then, for example, be used for cloning or subcloning nucleic acid molecules of interest. General classes of vectors of particular interest include prokaryotic and/or eukaryotic cloning vectors, expression vectors, fusion vectors, two-hybrid or reverse two-hybrid vectors, shuttle vectors for use in different hosts, mutagenesis vectors, transcription vectors, vectors for receiving large inserts, and the like.

It is also understood that the constructs described herein may contain a eukaryotic viral origin of replication, either in place of, or in conjunction with an amplifiable marker. These origins may be present in place of, or in conjunction with, an amplifiable marker. The presence of the viral origin of replication allows the integrated vector and adjacent endogenous gene to be isolated as an episome and/or amplified to high copy number upon introduction of the appropriate viral replication protein. Examples of useful viral origins include, but are not limited to, SV40 ori and EBV ori P. Vectors of the present invention can contain DNA sequences that exist in nature or that have been created by genetic engineering or synthetic processes.

The vector may also contain genetic elements useful for the propagation of the construct in micro-organisms. Examples of useful genetic elements include microbial origins of replication and antibiotic resistance markers.

C. Integration Cassettes

Integration cassettes (IC's) are the genetic constructs that are initially incorporated into cells to form the libraries and expression systems of the present invention. Incorporation of IC's is typically via non-homologous recombination at random loci throughout the cellular genome, as is the case for exogenously-derived nucleic acids lacking homology regions with genomic sequences, or site-directed recombination elements and/or enzymes. Randomly inserted also refers to “pseudo-random” insertion, where certain insertion sites are preferred over insertion generally into the endogenous DNA, provided the preference is not exclusive to a small subset of sites. Preferably preferential insertion into a subset of sites (in a pseudo-random context) should not exceed 40% of the rate found for sites outside the subset, more preferably 20% and most preferably not more than 10% over the random rate of insertion. Although integration at random genetic loci by IC's generally leads to stable transformants, the eukaryotic genome has regions where genetic expression is largely suppressed. Integration of an expression construct into one of these genetic “quiet” regions leads to suppressed expression from the construct. By allowing the expression level of the randomly integrated IC expression system to be evaluated prior to substitution with, and production of, a desired protein product, the IC's of the present invention allow for the rapid development of stable expression systems displaying desirable transcriptional and/or translational levels.

A feature of the IC's of the present invention that allows for the development of such expression systems is the exchangeable homeostatic reporter segment. As initially integrated, the IC contains an exchangeable reporter segment. This exchangeable segment contains at least one scorable homeostatic reporter element that allows an expression property, e.g. the expression level generated by the IC, to be quantitated. As homeostatic reporter element expression can be quantitated without adversely affecting cell viability, expression levels can be determined using one or a few cells, thereby alleviating the need to clonally expand transformants before analysis, speeding up the analysis. Once a transformant comprising an IC supporting a desired level of expression has been isolated, the present invention provides constructs and methods for replacing the exchangeable reporter segment with an exchangeable target segment containing a target element encoding the desired protein. Once the exchangeable target segment is in place, the IC should transcribe the target element at the same rate that was determined for the reporter segment. Speed of analysis is an important feature by itself. In other circumstances, speed may be essential, e.g., where replication may result in loss of phenotype, e.g., in hybridoma fusions the fusion products may delete the critical chromosomes encoding the relevant immunoglobulin genes before growth and characterization of the hybridoma is completed.

IC's are structurally defined as an exchangeable segment (e.g., exchangeable reporter segment, or ERS) comprising at least one scorable homeostatic reporter element operably linked to a promoter. Flanking the reporter element within the ERS is a pair of recombinase recognition sites. These sites can be specific for the same recombinase activity, or different recombinases, but they cannot be recombination-compatible with each other.

A transcriptional unit comprising the reporter element will normally include an operable 3′ termination sequence. The 3′ termination sequence can be optionally located within the ERS, or downstream from the ERS. Preferably, the 3′ transcriptional termination sequence is located downstream of the ERS, as this position ensures that an exchangeable segment swapped into the integration cassette is controlled by the same set of regulatory sequences as the reporter element originally displaced.

An IC can also comprise several other genetic elements to aid in selection, scoring or expression of the integrated cassette. For example, the IC can contain enhancer sequences and/or operator sequences to aid in transcriptional regulation. Additional transcriptional units can be incorporated into the IC to, e.g., add other scorable or selectable markers, or other expressed protein markers. Internal ribosome entry site (IRES) sequences also allow additional transcriptional expression, by allowing more than one protein to be expressed from a single mRNA transcript. IRES sequences are particularly useful for monitoring expression of transcripts of the present invention. By placing a scorable marker gene linked to an IRES sequence downstream from a target element to be expressed, expression of the target element can be determined by monitoring expression of the linked scorable marker (alternatively, the target element can be linked to the IRES sequence and placed downstream in the transcript from a scorable marker).

Still other genetic elements that can be included in an IC are secretory signal elements that direct secretion of transcription products to which they are linked, and tags, anchors or other genetic elements that would allow an expression product linked to them to be specifically identified, or bound to a desired substrate. Such genetic elements include HIS tags, small fluorescent proteins, antigenic sequences, transmembrane domains, GPI linkages, and enzymes that can convert their substrates into detectable products. These genetic elements necessarily must be incorporated into the IC in-frame with the target sequence that is to be secreted, tagged or anchored. The additional genetic element(s) can be placed within the exchangeable segment containing the target element, or outside the exchangeable segment. In the latter case, the additional genetic element(s) are retained in the integration cassette regardless of the nature or number of exchangeable segments swapped into the cassette. For this reason, placing these additional genetic elements outside of the exchangeable segment is preferred.

For purposes of the present invention, an IC can comprise either an exchangeable reporter or exchangeable target segment. Both types of exchangeable segments can contain a reporter element and/or a target element for the expression of a desired product, or incorporation of cloning sites within the IC. Exchangeable reporter segments of the present invention however, typically comprise a scorable homeostatic reporter element, whereas exchangeable target segments typically comprise a target element encoding a desired protein product, or cloning sites.

1. Regulatory Elements

Transcription and translation regulatory elements are included in the constructs of the present invention to initiate and control expression of the coding regions found in the integration cassettes and rec elements. Regulatory elements include promoters and 3′ termination sequences, enhancer sequences and the like. Generally, regulatory elements are chosen based upon the cell type and conditions under which the desired gene product is to be expressed and can be isolated from cellular or viral genomes. Assays for regulatory sequence functionality are available. Briefly, suitable regulatory sequences can be identified by, e.g., conducting expression tests in a suitable test cell line using a scorable reporter gene. The regulatory sequence to be tested is operably linked to the scorable reporter gene and an additional regulatory sequences required. The construct is then expressed in the test cell line and an assay performed to detect the scorable reporter.

Examples of cellular regulatory sequences include, e.g., regulatory elements from the genes encoding actin, metallothionein I, an immunoglobulin, casein I, serum albumin collagen, globin laminin, spectrin ankyrin, sodium/potassium ATPase, and tubulin. Examples of viral regulatory sequences include, e.g., regulatory elements from Cytomegalovirus (CMV) immediate early gene, adenovirus late genes, SV40 genes, retroviral LTRs, and Herpesvirus genes. Typically, regulatory sequences contain binding sites for transcription factors such as NF-κB, SP-1, TATA binding protein, AP-1, and CAAT binding protein. Functionally, the regulatory sequence is defined by its ability to promote, enhance, or otherwise alter transcription of an endogenous gene.

Positioning of regulatory sequences within an expression system is generally known and will depend upon the source of the regulatory sequence and the environment in which it will be used. Typically regulatory sequences are positionally orientated in the IC similar to that found in their native state. Re-positioning regulatory sequences from model arrangements can be routinely performed using the molecular biology methodology referenced hereinabove, and optimal positioning determined through routine experimentation.

Promoters

Promoters are regulatory elements that initiate transcription of coding regions and can be incorporated into the integration cassettes and rec elements of the invention. As described below, some promoter elements are also used to temporally control genetic expression. Suitable promoters include constitutive, inducible, tissue or organ specific, or developmental stage specific promoters which can be expressed in the particular cell type used in the present invention. The choice of the promoter depends upon the type of host cell to be employed for expressing a gene(s) under the transcriptional control of the chosen promoter. A wide variety of promoters functional in viruses, prokaryotic cells and eukaryotic cells may be employed in the present invention.

Exemplary constitutive promoters in mammals include the EF-1α promoter, viral promoters such as HSV, TK, RSV, SV40 and CMV promoters, and various housekeeping gene promoters, as exemplified by the β-actin promoter. Examples of suitable mammalian inducible promoters include promoters from genes such as cytochrome P450, heat shock protein, metallothionein, hormone-inducible, such as the estrogen gene promoter, and such like. Promoters that are activated in response to exposure to ionizing radiation, such as fos, jun and erg-1, are also contemplated. Exemplary tissue-specific promoters include promoters from the liver fatty acid binding (FAB) protein gene, specific for colon epithelial cells; the insulin gene, specific for pancreatic cells; the transphyretin, alpha. 1-antitrypsin, plasminogen activator inhibitor type I (PAI-1), apolipoprotein Al and LDL receptor genes, specific for liver cells; the myelin basic protein (MBP) gene, specific for oligodendrocytes; the glial fibrillary acidic protein (GFAP) gene, specific for glial cells; OPSIN, specific for targeting to the eye; and the neural-specific enolase (NSE) promoter that is specific for nerve cells.

Exemplary plant promoters include, for example: the CaMV 35S promoter (Odell, J. T., Nagy, F., Chua, N. H., Nature, 313:810-812 (1985)), the CaMV 19S (Lawton, M. A., Tierney, M. A., Nakamura, I., Anderson, E., Komeda, Y., Dube, P., Hoffman, N., Fraley, R. T., Beachy, R. N., Plant Mol. Biol., 9:315-324 (1987)), nos (Ebert, P. R., Ha, S. B., An. G., PNAS, 84:5745-5749 (1987)), Adh (Walker, J. C., Howard, E. A., Dennis, E. S., Peacock, W. J, PNAS, 84:6624-6628 (1987)), sucrose synthase (Yang, N. S., Russell, D., PNAS, 87:41444148 (1990)), α-tubulin, actin (Wang, Y., Zhang, W., Cao, J., McEhoy, D. and Ray Wu., Molecular and Cellular Biology, 12:3399-3406 (1992)), cab (Sullivan, T. et al., Mol. Gen. Genet, 215:431-440 (1989)), PEPCase (Hudspeth, R. L. and J. W. Grula., Plant Mol. Biol., 12:579-589 (1989)) or octopine synthase (OCS) promoters, the light-inducible promoter from the small subunit of ribulose bis-phosphate carboxylase (Khoudi, et al., Gene, 197:343 (1997)) and the mannopine synthase (MAS) promoter (Velten et al., EMBO J., 3:2723-2730 (1984); Velten & Schell, Nucleic Acids Research, 13:6981-6998 (1985)). Tissue specific promoters such as root cell promoters (Zhang & Forde, Science, 279:407 (1998); Keller, et al., The Plant Cell, 3(10):1051-1061 (1991); Conkling, M. A., Cheng, C. L., Yamamoto, Y. T., Goodman, H. M., Plant Physiol., 93:1203-1211 (1990)). Still other promoters are wound-inducible and typically direct transcription not just on wound induction, but also at the sites of pathogen infection. Examples are described by Xu et al., Plant Mol. Biol., 22:573-588 (1993); Logemann et al., Plant Cell, 1:151-158 (1989); and Firek et al., Plant Mol. Biol., 22:129-142 (1993).

Termination Sequences and Enhancers

3′ Termination sequences signal the transcriptional apparatus to cease transcription. In addition, termination sequences also mark 3′ cleavage and polyadenylation sites of the transcript; two events that are generally considered important in allowing the transcript to be further processed and/or translated into protein. 3′ termination sequences are generally chosen to match the host cell and preferably the promoter used in the IC. For example 3′ termination sequences of genes expressed in mammals are preferred in mammalian cells, plant sequences are typically preferred in plant cells and termination sequences from expressed fungal genes in fungi. This 3′ termination sequence preference holds regardless of the source of the coding sequence being expressed. More preferably the 3′ termination sequence is from a gene expressed in the same cell type as the host cell used in the present invention. Ideally, the 3′ termination sequence is taken from a gene expressed in the host cell itself. The present invention should not be limited by the nature of the polyadenylation sequence chosen. Examples of suitable 3′ termination sequences include, but are not limited to, those from the bovine growth hormone sequence, the simian virus 40 sequence and the Herpes simplex virus thymidine kinase sequence.

Enhancer sequences can be from any suitable source, but generally follow the preference pattern described above for 3′ termination sequences, albeit with less stringency as heterogeneity between enhancer sequences and cell type is tolerated well in terms of functionality than is corresponding heterogeneity of 3′ termination sequence and cell type.

In alternative preferred embodiments, the regulatory element may be or may contain an enhancer. In particularly preferred such embodiments, the enhancer is the cytomegalovirus immediate early gene enhancer. In alternative embodiments, the enhancer is a cellular, non-viral enhancer.

Internal Ribosome Entry Sites (IRES Sequences)

IRES sequences are included in the present invention to allow multi cistronic transcripts to be produced. This allows expression systems of the present invention to produce subunits of a molecular complex from a single transcriptional unit, or to readily incorporate selectable and/or scorable reporters into exchangeable segments without creating fusion proteins or the necessity of additional regulatory elements to control expression of the second gene.

Most eukaryotic and viral messages initiate translation by a mechanism involving recognition of a 7-methylguanosine cap at the 5′ end of the mRNA. In a few cases, however, translation occurs via a cap-independent mechanism in which an internal ribosome entry site (IRES) positioned 3′ downstream of the gene translated from the cap region of the mRNA is recognized by the ribosome, allowing translation of a second coding region from the transcript. This is particularly important in the present invention as, having identified a particularly valuable expression site within the cellular genome, an IRES sequence allows simultaneous expression of multiple proteins from a single genetic locus. A particularly preferred embodiment involves including coding sequences for both a desired recombinant product and a selectable or scorable marker within the same exchangeable segment. Successful recombination events are marked by both expression of the desired recombinant product and the easily detectable marker, facilitating selection of successfully transfected cells. Examples include those IRES elements from poliovirus Type I, the 5′UTR of encephalomyocarditis virus (EMV), of “Thelier's murine encephalomyelitis virus (TMEV) of “foot and mouth disease virus” (FMDV) of “bovine enterovirus (BEV), of “coxsackie B virus” (CBV), or of “human rhinovirus” (HRV), or the “human immunoglobulin heavy chain binding protein” (BIP) 5′UTR, the Drosophila antennapediae 5′UTR or the Drosophila ultrabithorax 5′UTR, or genetic hybrids or fragments from the above-listed sequences. IRES sequences are described in Kim, et al., Molecular and Cellular Biology 12(8):3636-3.643 (August 1992) and McBratney, et al., Current Opinion in Cell Biology 5:961-965 (1993). IRES sequences also allow a single target element to include coding sequences for multiple proteins. These coding sequences may encode the same protein, or different proteins e.g., the heavy and light chains of an antibody. By including coding sequences for multiple proteins in a single transcript, equivalent expression levels for the proteins can be obtained.

2. Scorable and Selectable Reporters

Various embodiments of the present invention utilize selectable and/or scorable reporter genes to indicate successful transformation (selectable reporters) or to measure expression rates generated by the recombinant system (scorable reporters). Depending on the purpose, the reporter can be located within the exchangeable segment of the integration cassette and under the control of the regulatory elements normally associated with the coding region of an exchangeable segment, or can be located outside the exchangeable segment and under the control of independent regulatory elements.

Exemplary selection systems include, but are not limited to, the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt cells, respectively. Also, antimetabolite resistance can be used as the basis of selection for dhfr, which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); hygro, which confers resistance to hygromycin genes (Santerre, et al., 1984, Gene 30:147); neomycin resistance (neo), hypoxanthine phosphoribosyl transferase (HPRT), puromycin (pac), dihydro-orotase glutamine synthetase (GS), carbamyl phosphate synthase (CAD), multidrug resistance 1 (mdr1), aspartate transcarbamylase, adenosine deaminase (ada), and blast, which confers resistance to the antibiotic blasticidin.

Recently, additional selectable genes have been described, namely trpB, which allows cells to utilize indole in place of tryptophan; hisD, which allows cells to utilize histinol in place of histidine (Hartman & Mulligan, 1988, Proc. Natl. Acad. Sci. USA 85:8047); and ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.). The use of visible reporters has gained popularity with such reporters as anthocyanins, β glucuronidase and its substrate GUS, luciferase and its substrate luciferin. Green fluorescent proteins (GFP) (Clontech, Palo Alto, Calif.) can be used as both selectable reporters (See, e.g., Chalfie, M. et al. (1994) Science 263:802-805.) and homeostatic scorable reporters. (See, e.g., Rhodes, C. A. et al. (1995) Methods Mol. Biol. 55:121-131.)

Physical and biochemical methods may also be used to identify or quantify expression of the gene constructs of the present invention. These methods include but are not limited to: 1) Southern analysis or PCR amplification for detecting and determining the structure of the recombinant DNA insert; 2) Northern blot, S-1 RNase protection, primer-extension or reverse transcriptase-PCR amplification for detecting and examining RNA transcripts of the gene constructs; 3) enzymatic assays for detecting enzyme activity, where such gene products are encoded by the gene construct; 4) protein gel electrophoresis, western blot techniques, immunoprecipitation, or enzyme-linked immunoassays, where the gene construct products are proteins; and 5) biochemical measurements of compounds produced as a consequence of the expression of the introduced gene constructs. Additional techniques, such as in situ hybridization, enzyme staining, and immunostaining, also may be used to detect the presence or expression of the recombinant construct in specific cells, organs and tissues.

Alternatively, the vector can contain a scorable homeostatic reporter, in place of or in addition to, the selectable reporter. A scorable homeostatic reporter allows the cells containing the vector to be isolated without placing them under drug or other selective pressures or otherwise risking cell viability. Examples of scorable homeostatic reporters include genes encoding cell surface proteins (e.g., CD4, HA epitope), fluorescent proteins, antigenic determinants and enzymes (e.g., β-galactosidase). The vector containing cells may be isolated, e.g., by FACS using fluorescently-tagged antibodies to the cell surface protein or substrates that can be converted to fluorescent products by a vector encoded enzyme.

Selection can also be effected by phenotypic selection for a trait provided by the target element product. The IC, therefore, can lack a selectable reporter other than the “reporter” provided by the endogenous gene itself. In this embodiment, activated cells can be selected based on a phenotype conferred by the expressed target element. Examples of selectable phenotypes include cellular proliferation, growth factor independent growth, colony formation, cellular differentiation (e.g., differentiation into a neuronal cell, muscle cell, epithelial cell, etc.), anchorage independent growth, activation of cellular factors (e.g., kinases, transcription factors, nucleases, etc.), expression of cell surface receptors/proteins, gain or loss of cell—cell adhesion, migration, and cellular activation (e.g., resting versus activated T cells). A selectable reporter may also be omitted from the construct when transfected cells are screened for target element products without selecting for the stable integrants. This is particularly useful when the efficiency of stable integration and expression is high.

The vector may contain one or more (e.g., one, two, three, four, five, or more, and most preferably one or two) amplifiable reporters to allow for selection of cells containing increased copies of the IC and/or enhanced expression of the target. Examples of amplifiable reporters include but are not limited to dihydrofolate reductase (DHFR), adenosine deaminase (ada), dihydro-orotase glutamine synthetase (GS), and carbamyl phosphate synthase (CAD).

3. TAG Sequences

TAG sequences are coding sequences located outside the exchange segment, but linked in-frame to the coding sequence of the exchange element. In this way, TAG sequences provide a convenient means for producing fusion proteins using the constructs of the present invention. Common fusion protein partners include glutathione S-transferase (“GST”), thioredoxin (“Trx”), maltose binding protein, C- and/or N-terminal hexahistidine polypeptide (His tag), polylysine and other binding molecules. Other embodiments are coupled to elements that allow the target product(s) to be easily identified, such as small fluorescent proteins, antigenic determinants(e.g., FLAG, CD4, HA), enzymes that produce detectable products and the like. Still other embodiments are coupled to signal elements that direct the target products to particular cellular compartments. Examples of signal elements include those directing proteins to cellular organelles or identify the protein for excretion, the secretory signal segments.

The fusion proteins may be engineered with a protease recognition site at the fusion point so that fusion partners can be separated by protease digestion to yield intact mature enzyme. Examples of such proteases include thrombin, enterokinase and factor Xa. However, any protease can be used which specifically cleaves the peptide connecting the fusion protein and the enzyme.

These properties are conferred upon the target products of the present invention by linking nucleic acids encoding the tag sequences in frame with the nucleic acid encoding the target product. The nucleic acid encoding the tag sequences can be linked 5′ or 3′ to the target product, and can be incorporated as part of the exchangeable segment or can be located outside the exchangeable segment, provided it is in frame with and part of the translational unit encoding the target product.

A preferred tag for fusion constructs of the present invention are spontaneously fluorescent proteins that retain their fluorescent properties when expressed in heterologous cells, which has provided biological research with new, unique and powerful tools (Chalfie et al, Science, 263:802 (1994); Prasher, Trends in Genetics, 11:320 (1995); WO 95/07463; Heim et al., Proc. Natl. Acad. Sci. USA, 91:12501 (1994)). As these proteins possess a compact structure and are relatively small in size (˜20-30 kDa), they can be linked directly to a target molecule, with or without an intervening linker, without significant effect on the functional properties of the target molecule. Linking the target products of the present invention is a preferred method of tagging target products, as the fluorescent proteins used in this manner serve as selectable and scorable homeostatic reporters of gene expression in addition to chromatic tags for the target product itself.

Secretory signal segments are typically N-terminal amino acid sequences capable of directing a polypeptide into the secretory pathway characteristic of eukaryotic cells. As these N-terminal amino acid sequences are typically cleaved as part of the secretory process, secretory signal segments useful in the practice of the present invention can easily be identified. For example the N-terminal amino acid sequence of a secreted protein can be compared with the amino acid sequence predicted from the cDNA sequence encoding the same protein. The N-terminal amino acids predicted by the cDNA sequence but missing from the excreted protein constitute a prospective signal sequence. A nucleic acid encoding this prospective signal sequence is potentially a secretory signal segment.

The prospective secretory signal segment can be tested for functionality by ligating it in-frame to a reporter gene, such as the coding sequence for alkaline phosphatase or green fluorescent protein. The resulting chimeric protein is then inserted into a suitable expression vector and transfected into a host cell where it can be expressed. Expression of the chimeric protein leading to appearance of the reporter gene product in the extracellular fluid indicates that the secretory signal segment is functional.

Methods for constructing the fusion proteins described in this section are exemplified in a number of the references noted in the “general recombination methods” section above. Transmembrane domains may be incorporated to link otherwise secreted proteins to the cell surface. Antibodies, normally secreted, may be cellularly associated to allow for FACS sorting.

D. Exchangeable Segments

Exchangeable segments structurally comprise one or more coding sequences, which may be repeated, flanked by recombinase recognition sites that allow compatible exchangeable segments in different constructs to be readily swapped with each other when in the presence of a suitable recombinase activity. Using exchangeable segments, a coding region can readily and precisely be placed under the expressional control of an integration cassette of the present invention.

In addition to the coding sequence(s), an exchangeable segment may also contain 3′ termination sequences operably linked to the coding sequence(s) and/or transcriptional enhancer sequences as well as other genetic elements included to enhance or regulate the level of transcription of the coding sequence(s). Preferably, exchangeable segments consist essentially of the coding sequences that could be exchanged together with any necessary regulatory elements. Most preferably, the exchangeable segments consist of only the coding sequences that are to be exchanged. Ideally, regulatory sequences will be fixed at the locus of IC integration, as a desired result of the invention is to produce stable expression systems that are capable of expressing a plurality of possible coding sequences at the same level. Fixing regulatory sequences at the locus of IC integration can be accomplished by placing such sequences outside the exchangeable segment.

The structural characteristics of exchangeable segments allow different coding regions to be swapped in and out of a single IC. This arrangement allows a user to first ascertain and then isolate cell transformants that possess an IC integrated at a genetic locus that supports a desirable property, e.g., level of transcription. The level of transcription is determined by measuring the amount of a scorable reporter encoded within the exchangeable reporter segment of the IC. Once isolated, the reporter segment can be replaced by a target segment comprising a target element encoding a desirable protein product. The exchange occurs through a site-specific recombination process that is dependent on specific characteristics shared by both the reporter and target segments and located within the recombinase recognition sites of the respective exchange segments. As the target elements of the exchange segments are in register with each other, exchange of exchangeable segments operably links the new target element with the regulatory elements of the integrated IC, introducing the new target element to the same genetic environment, e.g., transcriptional activity such as level, and under the same control as the previous target or reporter element.

1. Scorable Homeostatic Reporter and Target Elements

Scorable homeostatic reporter elements are coding sequences for scorable homeostatic reporters, and are included in the exchangeable reporter segment of the integration cassette to allow the determination of the expression level of the integration cassette at its genomic insertion site.

Target elements are structurally analogous to scorable homeostatic reporter elements in the sense that both are coding sequences located in an exchangeable segment of the invention. Target elements however need not be scorable, and comprise a coding region for a protein of interest. In addition, target elements may also comprise selectable or scorable reporters whose translation is controlled by an IRES sequence.

“Scorable homeostatic reporter element” refers to both genetic traits and the genes that encode the traits, typically, whose presence can be physically or chemically detected and quantified without adversely affecting the viability of the cell expressing the scorable homeostatic reporter element. For example, activity of an expressed enzyme can be scored by assaying for the enzyme activity. An example of a physically detectable trait is the fluorescence produced by green fluorescent proteins, which again can be measured and quantified, giving a determination of the amount of the fluorescent protein present, and hence expressed. Several exemplary scorable homeostatic reporters are listed above in the section “scorable and selectable reporter elements.” The scorable homeostatic reporter element need not contain only scorable genetic sequences, but may also encode exchangeable reporter genes that are selectable or otherwise act as a reporter element and detected without the need for quantification.

“Target elements” are nucleic acid sequences encoding a desired product. Examples of proteins with known activities include, but are not limited to, cytokines, growth factors, neurotransmitters, enzymes, structural proteins, cell surface receptors, intracellular receptors, hormones, antibodies, antisense and small inhibitory RNA's (snRNA's), and antigens, including viral antigens, proteases, plant growth factors, antibiotics, and transcription factors. These proteins often serve as useful biologics for which therapeutic activities exist, and high levels of expression for commercial production and manufacturing are desirable. A preferred product is a polypeptide of an antibody, including single chain antibodies, Fab and Fab′ fragments. Another preferred target element is a “polylinker.”

Polylinkers typically do not encode a protein product, but rather are short lengths of DNA that contain numerous different endonuclease restrictions sites located in close proximity. The presence of the polylinker is advantageous because it allows various expression cassettes to be easily inserted and removed, thus simplifying the process of making a construct containing a particular DNA fragment. Some embodiments of the invention have polylinkers comprising a nucleic acid sequence that is homologous with a portion of a nucleic acid sequence to be integrated into the construct. Such nucleic acid sequences are typically 5 to 200 bases long, more typically 10-100 bases long and most preferably 15-50 bases long. The important aspect of the homologous sequence is that it is of sufficient length and suitably free of interfering secondary structure so as to allow homologous recombination between the two homologous strands.

The invention encompasses expression of target elements both in vivo and in vitro. Therefore, cells transformed with the constructs of the present invention could be used in vitro to produce desired amounts of a protein or could be used in vivo to provide that gene product in the intact animal. Subsequent purification may be desired.

The proteins can be produced from either known, or previously unknown genes. Specific examples of known proteins that can be encoded by a target element and produced by the present invention include, but are not limited to, erythropoietin, insulin, growth hormone, glucocerebrosidase, tissue plasminogen activator, granulocyte-colony stimulating factor (G-CSF), granulocyte/macrophage colony stimulating factor (GM-CSF), macrophage colony-stimulating factor (M-CSF) interferon α, interferon β, interferon γ, interleukin-2, interleukin-3, interleukin-4, interleukin-6, interleukin-8, interleukin-10, interleukin-11, interleukin-12, interleukin-13, interleukin-14, TGF-β, blood clotting factor V, blood clotting factor VII, blood clotting factor VIII, blood clotting factor IX, blood clotting factor X, TSH-β, bone growth factor-2, bone growth factor-7, tumor necrosis factor, α-1 antitrypsin, anti-thrombin III, leukemia inhibitory factor, glucagon, Protein C, protein kinase C, stem cell factor, follicle stimulating hormone β, urokinase, nerve growth factors, insulin-like growth factors, insulinotropin, parathyroid hormone, lactoferrin, complement inhibitors, platelet derived growth factor, keratinocyte growth factor, hepatocyte growth factor, endothelial cell growth factor, neurotropin-3, thrombopoietin, chorionic gonadotropin, thrombomodulin, alpha glucosidase, epidermal growth factor, and fibroblast growth factor. The invention also allows the activation of a variety of genes expressing transmembrane proteins, and production and isolation of such proteins, including but not limited to cell surface receptors for growth factors, hormones, neurotransmitters and cytokines such as those described above, transmembrane ion channels, cholesterol receptors, receptors for lipoproteins (including LDLs and HDLs) and other lipid moieties, integrins and other extracellular matrix receptors, cytoskeletal anchoring proteins, immunoglobulin receptors, CD antigens (including CD2, CD3, CD4, CD8, and CD34 antigens), and other cell surface transmembrane structural and functional proteins. Other cellular proteins and receptors are known and may also be produced by the methods of the invention.

2. Recombinase Systems

The recombinase recognition sites that define the 5′ and 3′ boundaries of exchangeable segments give the site-specific recombination events that lead to segment exchange their site-specificity and their polarity. Recombination between two recombinase recognition sites will mormally only occur if the two sites are recognized by the recombinase as homologous sequences. By flanking the exchangeable segments with recognition sites that are not homologous, directionality can be impinged on the system. Moreover, if a target segment is flanked by recognition sites that are homologous to those flanking an exchangeable segment in an IC, the target segment recognition sites can undergo recombination with their homologous counterparts in the IC, leading to substitution of the target segment into the IC. Furthermore, if the recombination sites of the target segment are in the same 5′ to 3′ orientation relative to the target element as the recombination sites of the IC exchangeable segment, then the target element of the target segment will be operably linked to the IC regulatory sequences upon substitution. As the recognition sites frequently form part of the transcriptional unit encoding the target element of the invention, it is desirable that the recognition sites do not contain any sequence information that could adversely affect expression, or site-specific recombination. Ideally, the recognition sites should also be short to eliminate as many heterologous amino acids as possible in the product. To accomplish this goal, recognition site sequences are frequently engineered to enhance recombinational fidelity and/or efficiency, and to remove or alter sequences that could otherwise adversely affect expression. Techniques for performing recognition site engineering are discussed in greater detail below.

Several different recombinase systems can be used to achieve site-specific recombination leading to segment substitution, as described above. As noted above, a number of different site specific recombinase systems can be used in the present invention. These include, but are not limited to, the Cre/lox system of bacteriophage P1, the FLP/FRT system of yeast, the Gin recombinase of phage Mu, the Pin recombinase of E. coli, the Sin recombinase of Staphylococcus aureus and the R/RS system of the pSR1 plasmid. Two preferred site specific recombinase systems are the bacteriophage P1 Cre/10× and the yeast FLP/FRT systems. In these systems a recombinase (Cre or FLP) will interact specifically with its respective recombinase recognition sites (10× or FRT respectively) resulting in site-specific recombination at the recognition sites. The FLP/FRT system of yeast is the most preferred site specific recombinase system since it normally functions in a eukaryotic organism (yeast), and is well characterized.

Exemplary recombinase systems suitable for the present invention are also described in Hoess et al., Nucleic Acids Research 14(6):2287 (1986); Abremski et al., J. Biol. Chem. 261(1):391 (1986); Campbell, J. Bacteriol. 174(23):7495 (1992); Qian et al., J. Biol. Chem. 267(11):7794 (1992); Araki et al., J. Mol. Biol. 225(1):25 (1992); Paulsen et al., Gene 141(1):109-14 (1994); Rowland et al., Mol. Microbiol. 44(3):607-19 (2002)). Many of these belong to the integrase family of recombinases (Argos et al. EMBO J. 5:433-440 (1986); Landy, A. (1993) Current Opinions in Genetics and Devel. 3:699-707). A preferred system is the Cre/loxP system from bacteriophage P1 (Hoess and Abremski (1990) In Nucleic Acids and Molecular Biology, vol. 4. Eds.: Eckstein and Lilley, Berlin-Heidelberg: Springer-Verlag; pp. 90-109). The most preferred system is the FLP/FRT system from the Saccharomyces cerevisiae 2μ circle plasmid (Broach et al. Cell 29:227-234 (1982)). Both the FLP and Cre systems have relatively short sequences that serve as recombinase recognition sites (47 bp and 34 bp, respectively).

Other embodiments utilize group II introns as recombination recognition sites. Group II introns are mobile genetic elements encoding a catalytic RNA and protein. The protein component possesses reverse transcriptase, maturase and an endonuclease activity, while the RNA possesses endonuclease activity and determines the sequence of the target site into which the intron integrates. By modifying portions of the RNA sequence, the integration sites into which the element integrates can be defined. Target elements can be incorporated between the ends of the intron, allowing targeting to specific sites. This process, termed retrohoming, occurs via a DNA:RNA intermediate, which is copied into cDNA and ultimately into double stranded DNA (Matsuura et al., Genes and Dev 1997; Guo et al, EMBO J, 1997). Numerous intron-encoded homing endonucleases have been identified (Belfort and Roberts, 1997. NAR 25:3379). Such systems can be easily adopted for application to the methods described herein.

The FLP/FRT recombinase system has been demonstrated to function efficiently in eukaryotic cells, particularly plant cells. The recombination reaction is reversible and this reversibility can compromise the efficiency of the reaction in each direction. Altering the sequence of the recombinase recognition sites is one approach to remedying this situation. The recognition sites can be mutated in a manner that the product of the recombination reaction is no longer recognized as a substrate for the reverse reaction, thereby stabilizing the substitution event. Another approach to manipulate the system is based on mass action and the equilibrium of the catalyzed reaction. By including a large molar excess of target segment over integration cassette, the substitution of the target segment into the IC will be favored, effectively stabilizing the substitution event.

Assays for FLP recombinase activity are known and generally measure the overall activity of the enzyme on DNA substrates containing FRT sites. In this manner, a frequency of excision of the target sequence can be determined. For example, inversion of a DNA sequence in a circular plasmid containing two inverted FRT sites can be detected as a change in position of restriction enzyme sites. This assay is described in Vetter et al. (1983) Proc. Natl. Acad. Sci. USA 80:7284. Alternatively, excision of DNA from a linear molecule or intermolecular recombination frequency induced by the enzyme may be assayed, as described, e.g., in Babineau et al. (1985) J. Biol. Chem. 260:12313; Meyer-Leon et al. (1987) Nucleic Acids Res. 15:6469; and Gronostajski et al. (1985) J. Biol. Chem. 260:12328.

As was the case for the IC promoter discussed above, the promoter controlling the expression of the nucleotide encoding the recombinase may be constitutive, tissue specific or inducible, allowing for temporal and quantitative control over the expression of recombinase activity when required.

Exemplary inducible promoters include the heat shock promoter and the glucocorticoid system. Promoters regulated by heat shock, such as the promoter normally associated with the gene encoding the 70-kDa heat shock protein, can increase expression several-fold after exposure to elevated temperatures.

In the present invention, it may also be advantageous to link a nuclear transfer signal sequence to the recombinase gene. The nuclear transfer signal sequence accelerates the transfer of the recombinase into the nucleus, Daniel Kalderon et al., Cell, 39, 499-509 (1984).

Engineered Recombinase Recognition Sites and Other Nucleic Acid Sequences

In some embodiments, the recombinase recognition sites of the present invention (or other nucleotide sequence to be transcribed) should be engineered to ensure that coding regions of the integration cassette are properly transcribed and/or translated. Recombinase recognition sites of the present invention frequently form part of the transcriptional unit comprising the target element encoding the protein whose expression is sought. Wild-type recognition sites may however contain sequences that reduce the efficiency of transcription and/or translation of the desired product or the specificity of recombination reactions. For example, multiple stop codons in attB, attR, attP, attL and loxP recombination sites occur in multiple reading frames on both strands, so translation efficiencies are reduced, e.g., where the coding sequence must cross the recombination sites, (only one reading frame is available on each strand of loxP and attB sites) or impossible (in attP, attR or attL).

Accordingly, the present invention also provides engineered recombination sites that overcome these problems. For example, att sites can be engineered to have one or multiple mutations to enhance specificity or efficiency of the recombination reaction and the properties of product DNAs (e.g., att1, att2, and att3 sites); to decrease reverse reaction (e.g., removing P1 and H1 from attR). The testing of these mutants determines which mutants yield sufficient recombinational activity to be suitable for recombination subcloning according to the present invention. The site-specific recombination sequence can occasionally be mutated in a manner that the product of the recombination reaction is no longer recognized as a substrate for the reverse reaction, thereby stabilizing the integration or excision event.

Mutations can therefore be introduced into recombination sites for enhancing site specific recombination. Such mutations include, but are not limited to: recombination sites without translation stop codons that allow fusion proteins to be encoded; recombination sites recognized by the same proteins but differing in base sequence such that they react largely or exclusively with their homologous partners allowing multiple reactions to be contemplated; and mutations that prevent hairpin formation of recombination sites. Which particular reactions take place can be specified by which particular partners are present in the reaction mixture.

There are well known procedures for introducing specific mutations into nucleic acid sequences. A number of these are described in Ausubel, F. M. et al., Current Protocols in Molecular Biology, Wiley Interscience, New York (1989-1996) and other references noted in the “general recombination methods” section of this application.

The functionality of the mutant recombination sites can be demonstrated in ways that depend on the particular characteristic that is desired. For example, the lack of translation stop codons in a recombination site can be demonstrated by expressing the appropriate fusion proteins. Specificity of recombination between homologous partners can be demonstrated by introducing the appropriate molecules into in vitro reactions, and assaying for recombination products. Other desired mutations in recombination sites might include the presence or absence of restriction sites, translation or transcription start signals, protein binding sites, and other known functionalities of nucleic acid base sequences. Genetic selection schemes for particular functional attributes in the recombination sites can be used according to known method steps. Similarly, selection for sites that remove translation stop sequences, the presence or absence of protein binding sites, etc., can be easily devised by those skilled in the art.

Accordingly, the present invention provides a nucleic acid molecule, comprising at least one DNA segment having at least two engineered recombination sites flanking a Selectable marker and/or a desired DNA segment, wherein at least one of said recombination sites comprises a core region having at least one engineered mutation that enhances recombination in vitro in the formation of a Cointegrate DNA or a Product DNA.

While in the preferred embodiment the recombinase recognition sites differ in sequence and do not interact with each other, it is recognized that sites comprising the same sequence can be manipulated to inhibit recombination with each other. Such conceptions are considered and incorporated herein. For example, a protein binding site can be engineered adjacent to one of the sites. In the presence of the protein that recognizes said site, the recombinase fails to access the site and the other site is therefore used preferentially.

III. Cellular Transformation with Integration Cassettes

Transforming competent cells with the integration cassettes of the present invention can be accomplished using routine techniques. Briefly, a suitable vector comprising an integration cassette of the present invention is introduced to a competent cell. The cell is then incubated under conditions that allow non-homologous recombination between the vector and the genetic material of the cell. In this manner the entire vector is inserted into the cellular genetic material. As the entire vector, not simply the integration cassette, is inserted into the cellular genomic material, minimal vector sequences are preferable, preferably being between 500 bp and 500 kbp long, more preferably between 1 kbp and 100 kbp long and most preferably between 5 kbp and 50 kbp in length.

It should also be noted that non-homologous recombination events using the constructs of the present invention are essentially random events, with substantially equal probability of occurring anywhere in the genome. As different loci of the genome present different genetic (and biochemical) environments, these different loci exhibit differential expression levels for inserted constructs, including genetically “silent” regions. By producing a large number of transformants, each comprising an integration cassette at a different locus in the genome, the present invention allows for the determination of an optimal genetic locus for gene expression. Once identified, cells containing the integration cassette of the invention inserted at this optimal locus can be clonally expanded. Using the recombinase systems described herein, a coding sequence or polylinker can be inserted at this site of optimal expression. This exchange of transgene material can be repeated multiple times, with the effect of each transgene exchange benefiting from the optimal location of the insertion site.

A. Suitable Host Cells

The integration cassettes of the present invention can be used to transform a eukaryotic or prokaryotic cell for a variety of purposes including, but not limited to, over expression of target elements, dynamic protein interaction studies, reverse genomic studies and gene therapy. Cells used in this invention can be derived from eukaryotic species, including but not limited to mammalian cells (such as rat, mouse, bovine, porcine, sheep, goat, and human), avian cells, fish cells, amphibian cells, reptilian cells, plant cells, and yeast cells. Preferably, over expression of an endogenous gene or gene product from a particular species is accomplished by activating gene expression in a cell from that species. For example, to over express endogenous human proteins, human cells are used. Similarly, to over express endogenous bovine proteins, e.g., bovine growth hormone, bovine cells are used.

Preferred features of expressing cell lines include being an adventitious agent and/or infectious agent growing in virus and serum free medium, having fast growth and replication rates, and typically a small size and shear resistance. The cell lines also preferably have high but stable transcription and translation capacities, and are resistant to hypoxia. In certain circumstances, high transformation rates will be preferred.

Examples of useful vertebrate tissues from which cells can be isolated and activated include, but are not limited to, liver, kidney, spleen, bone marrow, thymus, heart, muscle, lung, brain, immune system (including lymphatic), testes, ovary, islet, intestinal, stomach, bone marrow, skin, bone, gall bladder, prostate, bladder, zygotes, embryos, and hematopoietic tissue. Useful vertebrate cell types include, but are not limited to, fibroblasts, epithelial cells, neuronal cells, germ cells (e.g., spermatocytes/spermatozoa and oocytes), stem cells, and follicular cells. Examples of plant tissues from which cells can be isolated and activated include, e.g., leaf tissue, ovary tissue, stamen tissue, pistil tissue, root tissue, tubers, gametes, seeds, embryos, and the like.

Preferred prokaryotic host cells include gram positive bacteria, e.g., a Bacillus cell, e.g., Bacillus alkalophilus, Bacillus amyloliquefaciens, Bacillus brevis, Bacillus circulans, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans and Streptomyces murinus, or gram negative bacteria such as E. coli and Pseudomonas sp. In a preferred embodiment, the bacterial host cell is a Bacillus lentus, Bacillus licheniformis, Bacillus stearothermophilus, or Bacillus subtilis cell. In another preferred embodiment, the Bacillus cell is an alkalophilic Bacillus.

Preferred eukaryotic host cells include CHO, myeloid, baby hampster kidney, COS, NSO, Hela and NIH323 cells, particularly, e.g., the monkey kidney CVI line transformed by SV40 (COS-7, ATCC CRL 1651); human embryonic kidney line (293, Graham et al. J. Gen Virol. 36:59 [1977]); baby hamster kidney cells (BHK, ATCC CCL 10); Chinese hamster ovary-cells-DHFR (CHO, Urlaub and Chasin, Proc. Natl. Acad. Sci. (USA) 77:4216, [1980]); mouse sertoli cells (TM4, Mather, Biol. Reprod. 23:243-251 [1980]); monkey kidney cells (CVI ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL51); TR1 cells (Mather et al., Annals N.Y. Acad. Sci 383:44-68 (1982)); human B cells (Daudi, ATCC CCL 213); human T cells (MOLT-4, ATCC CRL 1582); and human macrophage cells (U-937, ATCC CRL 1593). The cells can be maintained according to standard methods well known to those of skill in the art (see, e.g., Freshney (1994) Culture of Animal Cells, A Manual of Basic Technique, (3d ed.) Wiley-Liss, New York; Kuchler et al. (1977) Biochemical Methods in Cell Culture and Virology, Kuchler, R. J., Dowden, Hutchinson and Ross, Inc. and the references cited therein). Cultured cell systems often will be in the form of monolayers of cells, although cell suspensions are also used, especially for commercial production.

In a preferred embodiment, one or more reporter genes are used to identify those cells that are successfully transfected. The same or a different reporter gene can be expressed by the expression cassette expressing the dsRNA to provide an indication of actual dsRNA expression.

Host cells can be transformed with integration cassettes using suitable means and cultured in conventional nutrient media modified as is appropriate for inducing promoters, selecting transformants or detecting expression. Suitable culture conditions for host cells, such as temperature and pH, are well known. The concentration of plasmid used for cellular transfection is preferably titrated to reduce the likelihood of expression in the same cell of multiple vectors encoding different affector RNA molecules. Freshney (Culture of Animal Cells, a Manual of Basic Technique, third edition Wiley-Liss, New York (1994)) and the references cited therein provides a general guide to the culture of cells. Transduced cells are cultured by means well known in the art. See, also Kuchler et al. (1977) Biochemical Methods in Cell Culture and Virology, Kuchler, R. J., Dowden, Hutchinson and Ross, Inc. Mammalian cell systems often will be in the form of monolayers of cells, although mammalian cell suspensions are also used.

B. Transformation Methods

Integration cassettes, target segments and recombinase genes may be introduced into a host cell utilizing a vehicle, such as a viral vector, or by various physical methods. Representative examples of such methods include transformation using calcium phosphate precipitation (Dubensky et al., PNAS 81:7529-7533, 1984), direct microinjection of such nucleic acid molecules into intact target cells (Acsadi et al., Nature 352:815-818, 1991), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al., PNAS 89:6094, 1990), lipofection (Felgner et al., Proc. Natl. Acad. Sci. USA 84:7413-7417, 1989), microprojectile bombardment (Williams et al., PNAS 88:2726-2730, 1991), polycation compounds such as polylysine, receptor specific ligands, liposomes entrapping the nucleic acid molecules, spheroplast fusion whereby E. coli containing the nucleic acid molecules are stripped of their outer cell walls and fused to animal cells using polyethylene glycol, viral transduction, (Cline et al., Pharmac. Ther. 29:69, 1985; Curiel et al. (1991) Proc NatlAcad Sci USA 88:8850-8854; Cotten et al. (1992) Proc Natl Acad Sci USA 89:6094-6098; Curiel et al. (1992) Hum Gene Ther 3:147-154; Wagner et al. (1992) Proc Natl Acad Sci USA 89:6099-6103; Michael et al. (1993) J Biol Chem 268:6866-6869; Curiel et al. (1992) Am J Respir Cell Mol Biol 6:247-252; Harris et al. (1993) Am J Respir Cell Mol Biol 9:441-447, and Friedmann et al.,. Science 244:1275, 1989), and DNA ligand (Wu et al, J. of Biol. Chem. 264:16985-16987, 1989); Debs and Zhu (1993) WO 93/24640; Mannino and Gould-Fogerite (1988) BioTechniques 6(7): 682-691; Rose U.S. Pat. No. 5,279,833; Brigham (1991) WO 91/06309; and Felgner et al. (1987) Proc. Natl. Acad. Sci. USA 84: 7413-7414, as well as psoralen inactivated viruses such as AAV or Adenovirus.

Direct cellular uptake of oligonucleotides (whether they are composed of DNA or RNA or both) per se is presently considered a less preferred method of delivery because, in the case of siRNA and antisense molecules, direct administration of oligonucleotides carries with it the concomitant problem of attack and digestion by cellular nucleases, such as the RNAses. One preferred mode for administration of the expression cassettes of the present invention takes advantage of known vectors to facilitate the delivery of the expression cassette such that it will be expressed by the desired target cells. Such vectors include plasmids and viruses (such as adenoviruses, retroviruses, and adeno-associated viruses) (and liposomes) and modifications therein (e.g., polylysine-modified adenoviruses (Gao et al., Human Gene Therapy, 4:17-24 (1993)), cationic liposomes (Zhu et al., Science, 261:209-211 (1993)) and modified adeno-associated virus plasmids encased in liposomes (Phillip et al., Mol. Cell. Biol., 14:2411-2418 (1994)), as described supra.

Where the host cell is a plant cell, expression vectors may be introduced by particle mediated gene transfer. Particle mediated gene transfer methods are known in the art, are commercially available, and include, but are not limited to, the gas driven gene delivery instrument described in McCabe, U.S. Pat. No. 5,584,807, incorporated by reference. Alternatively, an expression cassette may be inserted into the genome of plant cells by infecting plant cells with a bacterium, including but not limited to an Agrobacterium strain previously transformed with the expression vector which contains an expression cassette of the present invention. (see, e.g., U.S. Pat. No. 4,940,838).

In some embodiments, restriction enzymes can be used to bias integration of integration cassettes to a desired site in the genome. For example, several rare restriction enzymes have been described which cleave eukaryotic DNA every 50-1000 kilobases, on average. If a rare restriction recognition sequence happens to be located upstream of a gene of interest, by introducing the restriction enzyme at the time of transfection along with the activation construct, DNA breaks can be preferentially upstream of the gene of interest. These breaks can then serve as sites for integration of the activation construct. The enzyme used cleaves in an appropriate location in or near the gene of interest and its site is under-represented in the rest of the genome or its site is over-represented near genes (e.g., restriction sites containing CpG). For genes that have not been previously identified, restriction enzymes with 8 bp recognition sites (e.g., NotI, SfiI, PmeI, SwaI, SseI, SrfI, SgrA1, PacI, AscI, SgfI, and Sse83871), enzymes recognizing CpG containing sites (e.g., EagI, Bsi-WI, MluI, and BssHII) and other rare cutting enzymes can be used.

Several methods for introducing restriction enzymes into cell are known in the art. (See for example, Yorifuji et al., Mut. Res. 243:121 (1990); Winegar et al., Mut. Res. 225:49 (1989); Pimplikar et al., J. Cell Biol. 125:1025 (1994); and Beckers et al., Cell 50:523-534 (1987)).

Following transfection, the cells are cultured under conditions, as known in the art. Culturing conditions may be modified to promote non-homologous recombination (e.g., transformation with an integration cassette), or homologous integration (e.g., when substituting exchangeable target segments).

C. Selecting Stable Transformants

Once an integration cassette is introduced into a cell, the cell is cultured under conditions designed to promote random integration of the cassette into the cellular genome through a non-homologous recombination process. The integration cassette will be incorporated into a statistically large number of sites within the resulting population of cells. As depicted in FIG. 1, the integration cassette can be comprised of selectable (and/or scoreable) reporters that can be located within or without the exchangeable reporter segment. Selection for the expression of these selectable reporters will isolate transformed cells. For example, the integration cassette illustrated in FIG. 1 contains both a CD4 and a Blast coding sequence, each transcribed from a different promoter. By culturing cells contacted with the integration cassette in a medium containing the antibiotic blasticidin. Cells transformed with the integration cassette of FIG. 1 will be blasticidin resistant and survive the treatment, while non-transformed cells will fail to proliferate.

The CD4 gene product of the FIG. 1 integration cassette can also be used to select transformed cells. The CD4 product is a cell surface receptor for HIV, and is highly antigenic. By using CD4-specific antibodies that are, example.g.,, fluorescently tagged, individual transformed cells producing the CD4 antigen can be identified and isolated (using for example, FACS sorting).

The use of reporter elements within the exchangeable reporter segment has several advantages over using selectable markers transcribed from separate promoters. These advantages include; 1. The ability to identify and isolate single cell transformants without clonal expansion; 2. Detection of expression driven by the promoter transcribing the exchangeable segment, and 3. In many cases, the ability to quantify the level of transcriptional activity supported by the promoter transcribing the exchangeable segment.

Selection of transformed cells is illustrated graphically in FIG. 2.

D. Quantitation and Sorting Methods Based On Expression Levels

In the context of the present invention, quantitation of genetic expression is preferably determined using scorable homeostatic reporters. With the exception of reporters capable of a calorimetric or phenotypic change in the cell, scorable homeostatic reporters are typically limited to those proteins that are either secreted (including fusion proteins coupled to secretory signal segments) or displayed on the cell membrane. Consequently, these preferred reporters are typically quantitated using calorimetric, microscopic or immunological assay methods.

Quantitative immunological assays are well known, and include immunoprecipitation, Western blot analysis (immunoblotting), ELISA and fluorescence-activated cell sorting (FACS). Shapiro (2002) Practical Flow Cytometry (4th ed.) Wiley & Sons; ISBN: 0471411256; McCarthy and MacEy (eds. 2002) Cytometric Analysis of Cell Phenotype and Function Cambridge Univ. Press; ISBN: 0521660297; Givan (2001) Flow Cytometry: First Principles (2d ed.) Wiley-Liss; ISBN: 0471382248; Radbruch (ed. 2000) Flow Cytometry and Cell Sorting (2d. ed.; Springer Lab Manual) Springer-Verlag; ISBN: 3540656308; and Ormerod (ed. 2000) Flow Cytometry: A Practical Approach (3d. ed.) American Chemical Society; ISBN: 0199638241.

Antibodies directed to reporter proteins can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found in, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

Once a cell has been transformed using the constructs and techniques of the present invention, it can be screened using a number of assays designed to detect the scorable and selectable reporter proteins. Depending on the characteristics of the reporters used (e.g., secreted versus membrane-associated) any or all of the assays described below can be utilized in addition to those previously mentioned. Typically, expression levels correlate with the intensity of the signal generated by the assay (e.g., the greater the detectable signal generated by the assay, the greater the expression level). Other assay formats known by those of skill in the art can also be used.

1. ELISA Assays

ELISA assays can be performed on secreted reporter proteins or reporters displayed on the cell membrane. By way of example, secreted proteins are quantified by adding cell-depleted growth media to microtiter wells that contain immobilized antibodies that specifically bind the reporter protein. Typically a specific or selective reaction will be at least twice background signal or noise and more typically more than 10 to 100 times background. After sufficient time has elapsed for the immobilized antibodies to bind the reporter protein, the residual media is removed and a second antibody specific for a different reporter epitope(s) and labeled with a detectable marker (e.g., a radiolabel, colored bead, enzyme or the like) is added. The immunocomplex formed is then washed to remove excess labeled antibody and the label developed. The expression level of the integration cassette will be proportional to the amount of developed label present in the assay. (See, e.g., Harlow & Lane, Antibodies, A Laboratory Manual (1988), for a description of immunoassay formats and conditions that can be used to determine specific immunoreactivity).

For systems comprising reporters displayed on the cell membrane, the assay can be performed in a similar manner using whole cells rather than secreted reporter proteins.

An alternative to immobilized antibodies are antibodies conjugated to magnetic beads. The magnetic bead-conjugated antibodies can be directly added to media containing reporter-expressing cells. Reporters, regardless of whether soluble or membrane-associated, can then be isolated by applying a magnet to the solution. The magnet isolates the magnetic bead-conjugated antibodies and anything bound to them. Labeled antibody can then be added to the isolated magnetic bead-conjugated antibodies and the resulting immunocomplex isolated and concentrated by repeating application of the magnet.

2. FACS Assay

The fluorescence-activated cell sorter (FACS) can be used to both screen for successful transformation and quantitate expression levels. FACS analysis also lends itself to analysis of reporters displayed on the cell surface, secreted, and those expressed intracellularly, provided the intracellular reporters are capable of producing a discernable fluorescent signal. If the reporter is a cell surface protein, then fluorescently-labeled antibodies that specifically bind the reporter are incubated with cells. If the reporter a secreted protein, then cells can be biotinylated and incubated with streptavidin conjugated to an antibody specific to the protein of interest (Manz et al., Proc. Natl. Acad. Sci. (USA) 92:1921 (1995)). Following incubation, the cells are placed in a high concentration of gelatin (or other polymer such as agarose or methylcellulose) to limit diffusion of the secreted protein. As protein is secreted by the cell, it is captured by the antibody bound to the cell surface. The presence of the protein of interest is then detected by a second antibody which is fluorescently labeled. For both secreted and membrane bound proteins, the cells can then be sorted according to their fluorescence signal. Fluorescent cells can then be isolated, expanded, and further enriched by FACS, limiting dilution, or other cell purification techniques known in the art.

A preferred reporter for FACS analysis are green fluorescent proteins (GFPs). GFPs are small proteins that can normally be expressed intracellularly without compromising cell viability. Proteins tagged with an intracellular GFP would be preferred over antibodies in FACS applications because such cells do not have to be incubated with the fluorescent-tagged reagent and because there is no background due to nonspecific binding of an antibody conjugate. GFP also does not require any substrates or cofactors.

Another feature of FACS analysis is that expression levels can be determined coincidentally with transformation efficiency, and prior to clonal expansion. This saves time, and reagents as only cell candidates known to support expression levels meeting a minimum threshold value are used for clonal expansion.

The level of expression of the reporter is generally proportional to the fluorescent signal, regardless of the technique used. Moreover, the techniques relating to FACS lend themselves to automated, high throughput assays using microtiter plates and fluorescent signal plate readers.

Methods for condicting studies using FACS techniques may be found in, e.g., Shapiro (2002) Practical Flow Cytometry (4th ed.) Wiley & Sons; ISBN: 0471411256; McCarthy and MacEy (eds. 2002) Cytometric Analysis of Cell Phenotype and Function Cambridge Univ. Press; ISBN: 0521660297; Givan (2001) Flow Cytometry: First Principles (2d ed.) Wiley-Liss; ISBN: 0471382248; Radbruch (ed. 2000) Flow Cytometry and Cell Sorting (2d. ed.; Springer Lab Manual) Springer-Verlag; ISBN: 3540656308; and Ormerod (ed. 2000) Flow Cytometry: A Practical Approach (3d. ed.) American Chemical Society; ISBN: 0199638241.

3. Western Blot (Immunoblot) Analysis

In relation to quantifying homeostatic reporters, western blot analysis is generally limited to analysis of secreted reporters, including fusion molecules comprising secretory signal segments. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the reporter. The antibodies may be directly labeled or alternatively may be subsequently detected using labeled antibodies (e.g., labeled sheep anti-mouse antibodies) that specifically bind to the anti-reporter antibodies.

4. Phenotypic Selection

In this embodiment for selection of transformants, cells can be selected based on a phenotype conferred by the reporter. Examples of phenotypes that can be selected for include proliferation, growth factor independent growth, colony formation, cellular differentiation (e.g., differentiation into'a neuronal cell, muscle cell, epithelial cell, etc.), anchorage independent growth, activation of cellular factors (e.g., kinases, transcription factors, nucleases, etc.), gain or loss of cell—cell adhesion, migration, and cellular activation (e.g., resting versus activated T cells). Isolation of activated cells demonstrating a phenotype, such as those described above, is important because the activation/silencing of an endogenous gene by the integrated construct or reporter expression is presumably responsible for the observed cellular phenotype. Thus, the endogenous gene may be an important therapeutic drug or drug target for treating or inducing the observed phenotype.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:3441 (1986)).

In certain embodiments of the invention, the target element comprises a coding sequence for a single protein. In other embodiments the target element comprises multiple coding sequences for a single protein. Still other embodiments comprise a target element having coding sequences for a plurality of different proteins. Finally, the invention contemplates integration of multiple integration cassettes into the same genome. Successful integration and target segment exchange can be determined by negative selection of the scorable markers. For example, should a target segment fail to exchange with a scorable reporter, such cells will retain the scorable reporter phenotype. In instances where multiple copies of the integration cassette, the scorable nature of the reporter phenotype allows a determination of the percentage of integration cassettes successfully undergoing recombinant incorporation of the target segment.

IV. Substitution of Exchangeable Segments by Site-specific Recombination

After selection for transformed cells and desired levels of transcriptional activity from the integration cassette in the selected expanded cells, an exchangeable target segment can be substituted into the integration cassette, replacing the exchangeable reporter segment. This is accomplished by introducing the target segment and a suitable recombinase activity to the transformed cell using one of the transformation techniques discussed above. The recombinase activity can reside on the same vector as the exchangeable target segments (e.g., see FIG. 3), or can be introduced to the cell through transformation with a separate vector (e.g., see FIG. 1). Each approach has distinct advantages. By including both the exchangeable target segment and the recombinase gene on the same vector, only a single vector need be taken up by the cell in a single step to incorporate the components necessary for segment substitution. By simplifying the process in this manner, the likelihood that a given cell will take up the necessary components is increased.

The alternative of transforming the cell with a target element and recombinase activity each located on separate vectors decreases the probability that each will be taken up by a given cell, but it does allow for control over the recombination event by delaying the process until the last component needed for the reaction is added. An alternative to placing the target segment and the recombinase on separate vectors is to place the recombinase gene under the control of an inducible promoter. The recombination event is then delayed until the cell containing of the necessary components is contacted by the inducing agent.

Still other alternative arrangements use pairs of recombinase systems that are not compatible. These alternative constructs were discussed previously in relation to recombinase recognition sites.

In certain embodiments of the invention, the target element comprises a coding sequence for a single protein. In other embodiments the target element comprises multiple coding sequences for a single protein. Still other embodiments comprise a target element having coding sequences for a plurality of different proteins. Finally, the invention contemplates integration of multiple integration cassettes into the same genome.

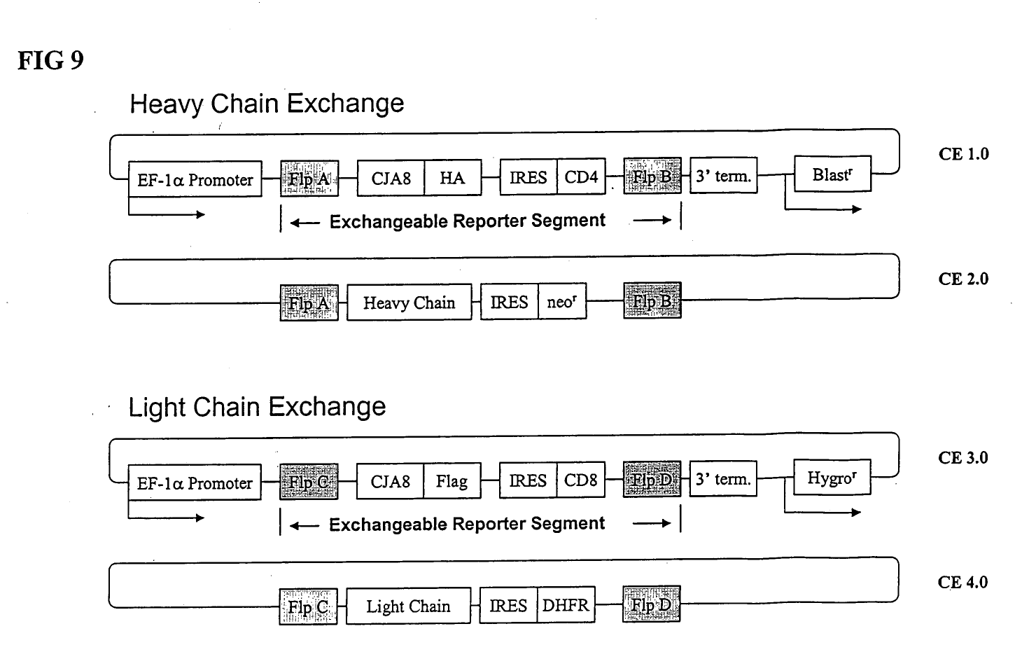

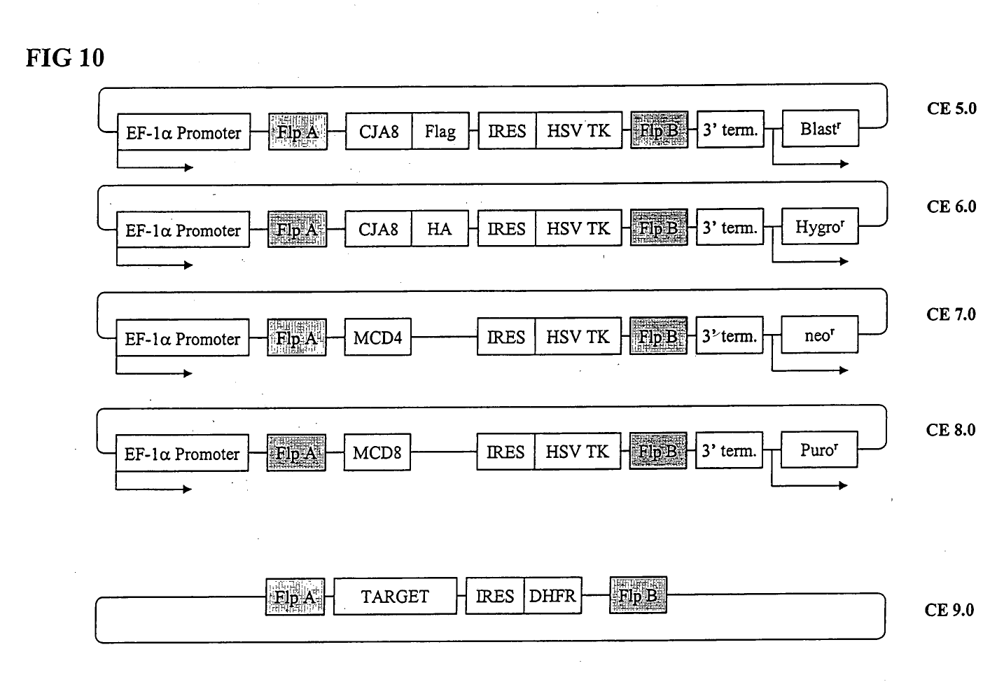

FIG. 9 depicts an exemplary set of integration cassettes and an exchangeable target segment for creating a production cell line comprising multiple integration cassettes. In this example (see also example 4, infra.), four integration cassettes are to be integrated into the cell (CE 5.0-8.0). Note that each of these integration cassettes has a different selectable marker transcribed from an independent promoter and located outside the recombinase recognition sites. (i.e., Blast^{r}, Hygro^{r}, neo^{r} and puro^{r}, respectively). These selectable markers allow for the selection of cells incorporating all or a subset of the integration cassettes. Second, each of the scoreable homeostatic reporter elements contains a scoreable marker (i.e., HSV TK). This scorable marker allows monitoring of both the number of integration cassettes initially integrated and the number of target elements successfully transferred into the integration cassette by site-specific recombination. The both characteristics are monitored by detecting the level of HSV TK expression. I.e., after transfection with the exchangeable target segment and a suitable recombinase activity, only HSV TK-cells have successfully replaced the reporter element with the target element.

Finally, note the use of the IRES sequence in FIG. 9. In the example depicted, the IRES sequence is used to create a polycistronic segment comprising a scorable reporter and an exchangeable reporter gene. IRES sequences can also be used to create target elements comprising multiple copies of a coding sequence of interest, or target elements comprising multiple transcription units.

As noted above, substitution of the target segment into the integration cassette can be driven to completion through a number of techniques. For example, the recombinase recognition sites of the integration cassette and/or the target segment can be genetically modified, such that they are not recognized by the recombinase after undergoing a recombination event with a target segment or integration cassette recognition site, respectively. More simply, a cell can be transformed with target segment nucleic acid in a molar excess relative to integration cassette nucleic acid.

A feature of the invention is that once the expression level supported by the promoter of an inserted integration cassette is determined, another target element placed under the control of that promoter will be expressed at the determined expression level. Moreover, using the techniques described above, virtually complete substitution of exchangeable segments can be achieved.

Successful substitution can be confirmed through selection processes analogous to those discussed above. For example, a selectable reporter different from that used in the integration cassette can be included in the exchangeable target cassette. This selectable reporter can be included in the same transcriptional unit as the target element or part of a separate transcriptional unit. In the former case, the “downstream” coding segment is typically operably linked to an IRES sequence, allowing independent translation of the respective coding regions.

An alternative to the selective marker approach discussed in the previous paragraph is selection of a phenotypic trait either associated with the target element itself, or lost from the integration cassette as a result of the recombination event that substitutes the target segment into the integration cassette (i.e., a phenotypic trait encoded in the exchangeable reporter cassette lost from the integration cassette upon recombination with the target segment), as discussed previously. Exemplary constructs that allow for this type of selection are depicted in FIG. 3.

V. Expression Systems for Multisubunit Complexes

Many important proteins, including enzymes, exist in multi-subunit complexes comprising more than one polypeptide chain. Exemplary multi-subunit complexes include antibodies, cell receptors, hormones, structural proteins and the like. In order to develop clonal cell populations capable of producing heterologous multi-subunit complexes, it is preferable to have each subunit of the complex expressed at a level in proportion to the molar ratio of other subunits as they appear in the complex. Expression systems of the present invention provide this feature.

By way of example, typical antibodies consist of two heavy chains and two light chains held together by disulfide bonds. In order to ensure that a recombinant cell can produce this preferred structure, the heavy and light chains of the antibody should be produced in an equimolar ratio. To accomplish this using the compositions and methods of the present invention, competent cells are first transformed with an integration cassette comprising a first scorable homeostatic reporter element, and transformants selected based on suitable expression of the homeostatic reporter as discussed herein.

The selected transformants are then transfected with a second integration cassette comprising a second homeostatic reporter element. Dually transformed cells are then selected based on a comparison of the expression levels determined for the first and second homeostatic reporters. In this instance, quantitatively equivalent expression levels are desired, as the two chains making up the preferred antibody structure are present in equimolar amounts. This scheme for producing transformants comprising dual integration cassettes is illustrated in FIG. 4.

Similarly, this can be repeated for multiple additional reporters. Alternatively, new sites may be evaluated for expression with the same reporters flanked with the same or different or recombinase.

By carefully controlling the conditions used in transforming the cells, it can be ensured that only a single copy of each integration cassette will be present in each cell. To ensure that only one heavy chain and one light chain are substituted into the respective integration cassettes, incompatible recombinase recognition sites are used to construct each integration cassette, as depicted in FIG. 5.

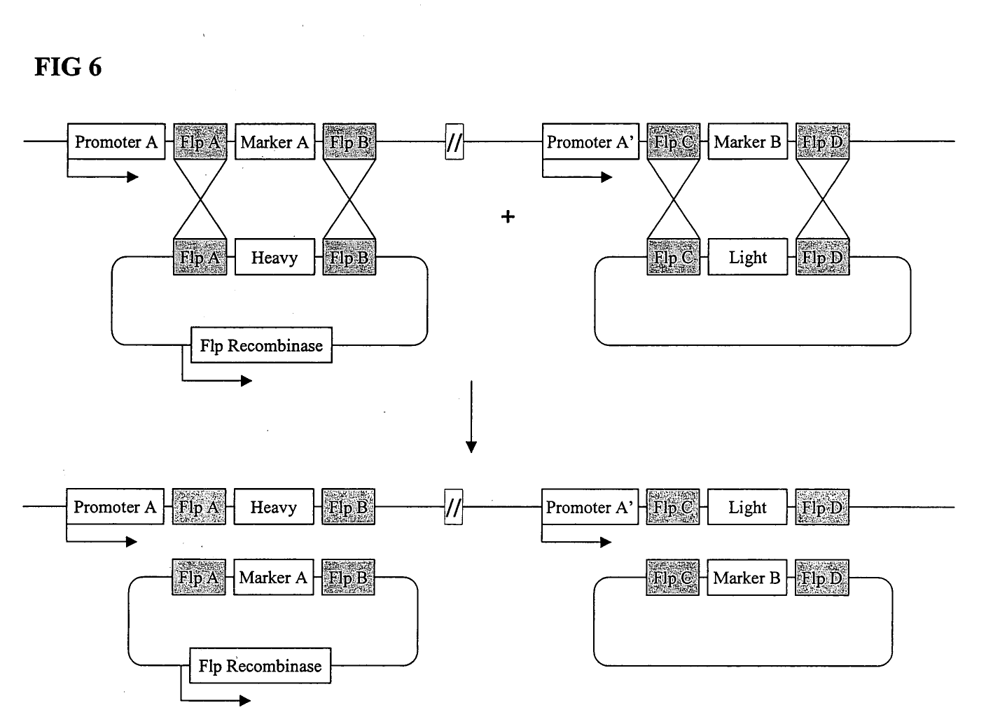

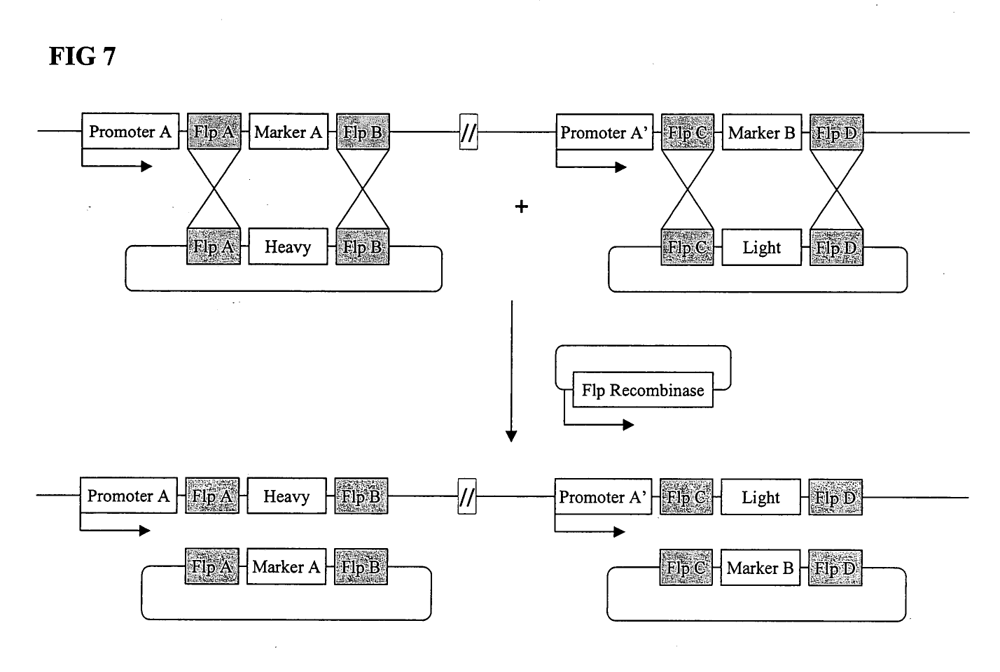

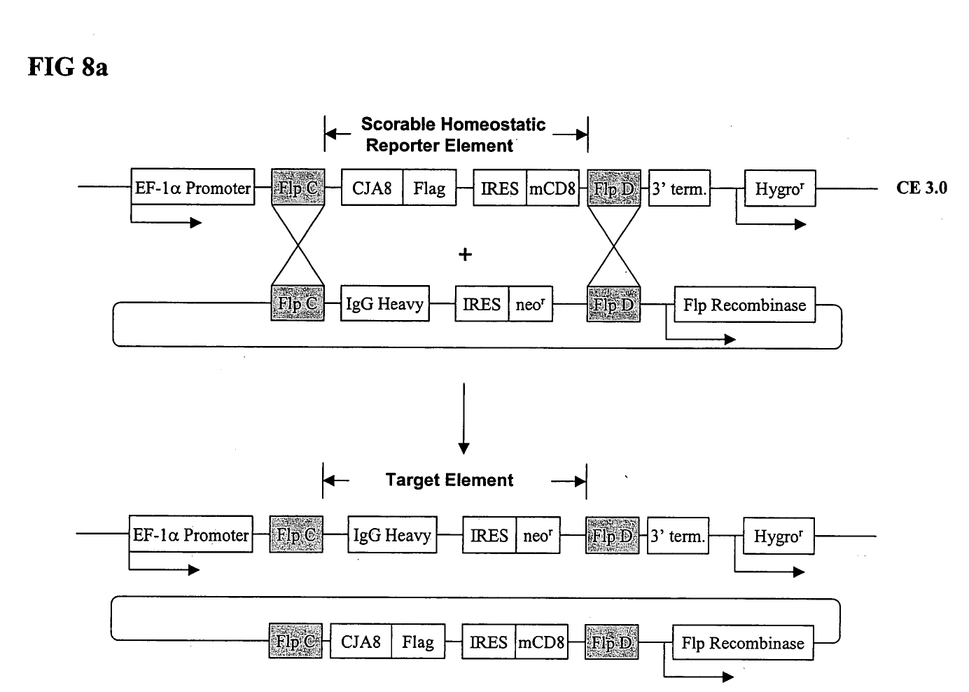

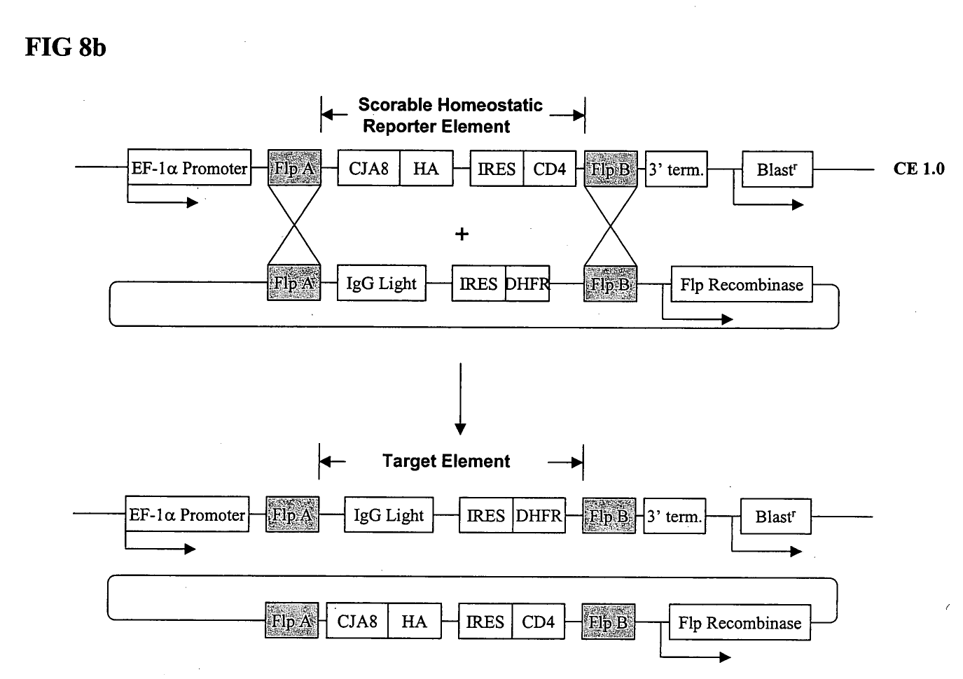

Selected transformants comprising the dual integration cassettes are then transformed with exchangeable target segments comprising two target elements, one consisting of the coding region for the antibody heavy chain and one consisting of the coding region for the antibody light chain, and a suitable recombinase activity. The presence of these components in the cell results in the cell simultaneously comprising an expression construct for an antibody heavy chain and an expression construct for an antibody light chain, each construct expressing its target element at a rate equivalent to that of the other construct. The lower panel of FIG. 5 depicts this result. FIGS. 6 and 7 illustrate other formats leading to the same result.

A particular feature of FIG. 5 is the presence of a TAG sequence at the 3′ end of the heavy chain integration cassette transcriptional unit. This TAG sequence is in frame with the target element (e.g., the heavy chain coding sequence) and can encode molecular reporter or marker proteins, anchors or binding proteins, as discussed herein above. Thus the constructs of the present invention afford the practitioner the ability of constructing novel recombinant expression systems, including expression systems for multi-subunit complexes that are heterofunctional. By way of example, the TAG sequence allows the practitioner construct an antibody that is His tagged simply by including a TAG sequence for six histidine residues. Such tag m y be incorporated into one of several copies of a particular gene.

VI. Expression Libraries

Also provided in the invention are nucleic acid libraries for genomic or cDNA production and expression, and the construction of expression libraries suitable for producing a host of useful variant proteins, such as monoclonal antibodies, heterofunctional antibodies, tagged reagents and labeled expression systems for interaction studies. These nucleic acid libraries are made up of a plurality of individual expression systems comprising at least one integration cassette where each distinct constituent member of the library has a target element consisting of a different nucleic acid portion or component, e.g., genomic fragment, cDNA, of an original whole nucleic acid library, i.e., fragmented genome, cDNA collection generated from the total or partial mRNA of an mRNA sample, etc. In other words, the libraries of the subject invention are nucleic acid libraries cloned into integration cassettes, where the nucleic acid libraries include, but are not limited to, genomic libraries, cDNA libraries, etc. Specific libraries of interest include, but are not limited to: Human Brain Poly A+ RNA; Human Heart Poly A+ RNA; Human Kidney Poly A+ RNA; Human Liver Poly A+RNA; Human Lung Poly A+ RNA; Human Pancreas Poly A+ RNA; Human Placenta Poly A+ RNA; Human Skeletal Muscle Poly A+ RNA; Human Testis Poly A+ RNA; and Human Prostate Poly A+RNA. Human, rabbit and mouse spleen and lymph node libraries and the like are also contemplated.

Of particular interest are libraries comprising variable sequences that affect functionality. Exemplary libraries of this type include, but are not limited to libraries of antibodies, Fab fragments, Fab′ fragments, single-chain antibodies, T-cell receptors, heterovalent antibodies, mutated enzymes, including G-protein coupled receptors and multi-subunit enzymes and hormones, antisense RNA sequences and siRNAs.

Variable sequences of the library members are preferably synthesized chemically by including all four bases in those synthesis cycles where randomized sequence is desired. Variable sequences are also preferably flanked by nucleotides of known sequence that become the 3′ end sequences for the promoters of the dual promoter system when the randomized dsRNA coding sequences are ligated into the expression cassette. Methods for incorporating synthetic nucleic acids into coding regions is discussed in Sambrook et al., Molecular Cloning: A Laboratory Manual; Cold Spring Harbor Laboratory, Cold Spring Harbor, N.Y. (1989); Ausubel et al., supra, as well as other references noted herein above.

Alternatively, mutant coding sequences for use as target elements in the present invention can be generated. Exchangeable target segments can then be used to substitute these mutant sequences into integration cassettes with known expression levels to test the effects of the mutation(s).

Libraries constructed according to the methods of the present invention also permit the rapid exchange of either individual clones of interest, groups of clones or potentially an entire cDNA library to a variety of expression systems comprising integration cassettes. The entire library may be transferred (using either an in vitro or an in vivo recombination reaction) into an expression vector modified to contain an integration cassette. This solves an existing problem in the art, in that there is no way, using existing vector systems, to exchange just the inserts in a library made in one expression vector en masse (i.e., as an entire library) to a different expression vector.

VII. Harvesting Expression Products

Expression products encoded in target elements and produced using the present invention can be harvested and purified. These methods include chromatographic techniques such as gel filtration, and ion exchange chromatographies (See, e.g., Hochuli, Chemische Industrie, 12:69-70 (1989); Hochuli, Genetic Engineering, Principle and Methods, 12:87-98 (1990), Plenum Press, N.Y.; and Crowe, et al. (1992) QIAexpress: The High Level Expression & Protein Purification System, QIAGEN, Inc. Chatsworth, Calif.), immunochemical techniques such as affinity chromatography and immunoprecipitafion, tagging techniques using, for example his tag, and epitope tagging, preferably using the TAG sequence feature of the integration cassette discussed above and depicted in FIG. 5. Electrophoresis and other techniques, such as those discussed in Schagger et al., Anal. Biochem., 166:368-379 (1987)); Scopes, Protein Purification: Principles and Practice (1982); Ausubel, et al. (1987 and periodic supplements); Current Protocols in Molecular Biology; Deutscher (1990) “Guide to Protein Purification” in Methods in Enzymology vol. 182, and other volumes in this series; and manufacturers' literature on use of protein purification products, e.g., Pharmacia, Piscataway, N.J., or Bio-Rad, Richmond, Calif.; and Sambrook et al., supra) can also be used.

VIII. Uses

In addition to the libraries discussed above the present invention is also useful in performing gene therapy techniques, developing novel therapeutics, studying protein/protein interactions and the like.

A. Development of Therapeutics

Libraries constructed according to the present invention can be used to screen for novel therapeutics. Recombinant products produced by the libraries can used to treat cells and the cellular response observed using high throughput techniques known in the art. Once identified, the integration cassette constructs of the invention can be used to produce and optionally tag recombinant products displaying interesting properties. For example, a recombinant product useful in arresting HIV production in an infected cell can be tagged with a CD4 Fab fragment using the TAG sequence feature of the present invention, thereby directing the recombinant product to HIV infected cells.

B. Gene Therapy

The integration cassettes of the present invention can also be used to create expression systems in cell lines modeling disease states. Expression libraries of the present invention comprising potential therapeutics can then be constructed using these model cell lines. In addition to expression of libraries of potentially therapeutic proteins, expression of potential antisense and siRNA sequences is also envisioned. Once identified, effective nucleic acids can be recovered from the integration cassettes using the disclosed recombinase system and routine recombinant molecular biological techniques. These effective nucleic acids can then be inserted into appropriate expression and delivery systems, including viral vectors, for use in gene therapy techniques.

Similar techniques to those noted above can be used to create transgenic plants. In addition to plant viral vectors, symbiotic bacteria, such as Agrobacterium sp. can be used both in creating the screening library and introducing nucleic acid sequences identified by the library as useful.

C. Study of Protein-protein Interactions

The expression systems of the present invention also find use in the study of protein-protein interaction. For example, by expressing two proteins in a cell comprising dual integration cassettes, the ability of the two proteins to interact can be studied in a manner reminiscent of the yeast two-hybrid system. Unlike the yeast two hybrid system however, the present invention allows the a eukaryotic protein complex to be expressed and studied in a more “natural” cellular environment, including possible expression in of cell types normally expressing the complex.

By way of example FRET studies can easily be performed using the present invention. A dual integration cassette expression system that includes the TAG sequence feature is first constructed in a cell line of choice. The TAG sequences of the integration cassettes consist of fluorescent proteins with overlapping excitation and emission spectra suitable for FRET studies. Using the recombinase systems of the invention, a library of potential binding partners is then constructed. Using fluorometric techniques known in the art, the library can then be screened for FRET activity in a high throughput manner. Thus the present invention addresses an additional shortcoming of the prior art: the need for a rapid, convenient two hybrid-type assay using cellular systems other than yeast.

D. Commercial Production Cell Lines

The present invention also includes production cell lines for the producing biologics and enzymes. Producion cell lines typically comprise multiple copies of the transcribable coding sequence of the protein to be produced. The usual way of including additional copies of an expressed sequence is to place all of the copies of the coding sequence for the protein to be produced in the target segment. Each coding sequence may be included in its own transcriptional unit, or each additional coding sequence may be under translational control of an IRES sequence. Alternatively, multiple copies of an integration cassette having the same recombinase recognition sites may be integrated into the cell (See FIG. 9 and example 4), as described earlier and infra.

The present invention has great value in dramatically shortening the time necessary to get a highly efficient production cell line from the initial genetic isolation to research level production, and subsequently into GMP production. The highly efficient and rapid identification of a characterized high efficiency commercial production grade cell line allows early production for early critical studies to establish therapeutic viability.

As such, one advantageous feature of these cell lines is high production yields from the earliest stages of development. Using the same cell line for initial studies as later development minimizes the disruptions and modifications in production which can slow a therapeutic development program.

The present invention provides reproducible and defined cell lines, particularly useful for commercial production purposes. The defined genetics, limited variability across cell lines, and fast selection are favorable features for this application.

Other advantageous features include freedom adventious and infection agents, e.g., viruses, high growth density and viability in the absence of serum and growth factors of animal origin (which introduce the risk of infectious agents), fast expansion and growth rates, robust cell properties under severe environmental conditions found in a production fermenter (e.g., properties of high cell density, viability, transcription, translation, protein folding, secretion, and overall protein production), shear resistance, homogeneous glycosylation under production conditions (e.g., which may exist within a large fermenter), and hypoxia resistance. See, e.g., Simonsen and McGrogan (1994) “The Molecular Biology of Production Cell Lines” Biologicals 22:85-94; Bendig (1988) “The Production of Foreign Proteins in Mammalian Cells” Genetic Engineering 7:91-127; Scheper, et al. (eds. 2000) New Products and New Areas of Bioprocess Engineering (Advances in Biochemical Engineering/Biotechnology, 68) Springer-Verlag; ISBN: 3540673628; Flickinger and Drew (eds. 1999) The Encyclopedia of Bioprocess Technology: Fermentation, Biocatalysis, and Bioseparation (Wiley Biotechnology Encyclopedias) Wiley & Sons; ISBN: 0471138223; and Lydersen, et al. (eds. 1994) Bioprocess Engineering Wiley-Interscience; ISBN: 0471035440. Starting cell lines can be selected for favorable properties in initial lines for development into systems as provided herein.

IX Kits

Kits are also provided for the practice of the present invention. Kits typically at least include an integration cassette, an exchangeable target segment and a recombinase that recognizes the recombinase recognition sites of the integration cassette and the target segment. The subject kits may further include other components that find use in practicing the invention, e.g., suitable vectors; reaction buffers, positive controls, negative controls, etc.

In addition to the above components, the subject kits will further include instructions for practicing the invention. These instructions may be present in the in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

Kits for production cells are also contemplated by the present invention. These typically at least include a sample of the production cell line and instructions for their growth and use. The kits may additionally contain antibiotic dosages for selection, antibodies for tagging and/or growth media to culture the cells. Other kits optionally comprise chromatography resins for purification of products and, reagents for performing control applications.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of noncritical parameters that could be changed or modified to yield essentially similar results.

### EXAMPLES

### Example 1

### Transformation of Chinese Hamster Ovary (CHO) Cells With an Integration Cassette

A pCE 1.0 CJA8 integration cassette was transfected into a CHO cell line by mixing 5 μg of purified vector DNA with 15 μl of Fugene transfection reagent and added to culture media containing 2×10⁶ cells on a 150 mm dish. After overnight incubation, cells were split (1:15) into new media supplemented with 2.5 μg/ml blasticidin. This selective media was changed every third day for two weeks. This selection resulted in several hundred colonies of about 1000 cells that had successfully integrated the vector.

The blasticidin resistant cells were removed from the plate with a PBS/EDTA solution and mixed to create a single cell suspension. The cells were then stained with an anti-CD4 antibody that had been labeled with a fluorescent dye (FITC). The stained cells were washed with PBS and run through a sterile FACS sort. The brightest 0.5% of the cells were collected and cloned by limiting dilution. The cells were re-checked for CJA8 expression.

The CE1.0 CJA8 integration cassette has one promoter driving expression of both the CJA8 exchangeable reporter element and the scorable reporter gene, CD4, the latter operably linked to an internal ribosome entry site (IRES). This construct allows each clone to express both the CD4 scorable reporter and the exchangeable reporter element at high levels.

### Example 2

### Exchanging a Reporter Segment for a Target Segment Using the Flp Recombinase System

A single clone from example 1 was expanded and transfected with plasmids containing an Flp recombinase expression cassette and the CE 2.0 BFH8 exchangeable target segment. The Flp recombinase mediated exchange of the CE 2.0 BFH8 exchangeable target segment for the exchangeable reporter segment in the integration cassette pCE1.0CJA8. After overnight incubation, the transfected cells were split (1:15) and G418 added to a concentration of 500 μg/ml. The cells were cultured in media containing 500 μg/ml G418 for two weeks, with media changes every three days. Under these conditions, cells that had successfully integrated the CE 2.0 exchangeable target segment were neo/G418 resistant and formed small colonies under the selective growth conditions.

Clones isolated in the manner described above were of two types. Most clones had successfully exchanged segments and were G418 resistant/CD4 negative. These were the desirable clones and were expressing the new target element at high levels. Some clones however had randomly integrated the CE 2.0 exchangeable target segment and were G418 resistant/CD4 positive. These two possibilities were distinguished using a CD4-ELISA assay.

### Example 3

### Constructing an Antibody Library

For a light chain gene or library we will start by transfecting the pCE 3.0 CJA8 vector into a cell line containing the pCE1.0 vector at a highly expressed site. So 5 ug of purified vector DNA will be mixed with 15ul of the Fugene transfection reagent and added to the culture media of 2×10⁶ cells on a 150 mm dish. The following day the cells will be split (1:15) and hygromycin will be added to the appropriate concentration for selection (200 ug/ml). This selective media will be changed every third day for two weeks. At this point cells that have successfully integrated this second vector will be blasticidin and hygromycin resistant and will have grown into colonies containing about 1000 cells. There will be several hundred colonies on the plate.

The cells will be removed from the plate with a PBS/EDTA solution and mixed to create a single cell suspension. The cells will then be stained with an anti-CD8 antibody that has been labeled with a fluorescent dye (FITC). The cells will then be washed with PBS and run through a sterile FACS sort. The brightest 0.5% of the cells will be collected and cloned by limiting dilution. Each of these clones will be expressing the surface CD4 and CD8 markers, as well as, the exchangeable reporter gene (CJA8) at high levels. The CE3.0 CJA8 vector is set up so that one promoter drives expression of both the CJA8 exchangeable reporter gene and the scorable reporter gene, CD8. Thus a single transcript encodes two coding regions that are linked via an internal ribosome entry site (IRES).

A single clone will be chosen for further manipulation. Cells from this clone will be expanded and transfected with plasmids containing an Flp recombinase expression cassette and the CE 2.0 heavy chain and CE 4.0 light chain vectors. The Flp recombinase will mediate the exchange of the expression cassette(s) in CE 2.0 heavy chain for the cassette from pCE1.0CJA8, which was integrated in the cell's genome in step one. It will also mediate the exchange of the CE4.0 light chain cassette(s) for the pCE3.0 CJA8 cassette integrated in step 2 above. The day after transfection the cells will be split (1:15) and G418 (500 ug/ml) and methotrexate will be added to an appropriate concentration for selection. This selective media will be changed every three days and after two weeks, cells, which have successfully integrated both the CE 2.0 and the CE4.0 cassettes, will be G418 resistant and methotrexate resistant. These cells will have formed small colonies under these selective growth conditions. These clones will be of several types. Most of the clones will have successfully exchanged both cassettes and will be G418 resistant and CD4 negative, as well as, methotrexate resistant and CD8 negative. These are the desirable clones and will be expressing antibodies at high levels. Some clones will have randomly integrated one or more of the exchange vectors and will be resistant to both drugs, but will still be expressing either CD4 or CD8 or both. The desirable cells can be separated from the population using the FACS and sorting for CD4/CD8 double negative cells. These cells will be expressing heterodimeric antibodies at high levels. They can be either cloned at this point or, in the case of an antibody library, the cells can be screened for antibodies with desirable properties.

1) Hoogenboom, H. R., J. D. Marks, A. D. Griffiths, G. Winter. Building antibodies from their genes. Immunol. Rev. 130: 41-68 (1992).

2) Marks, J. D., M. Tristrem, A. Karpas, G. Winter. Oligonucleotide primers for polymerase chain reaction amplification of human immunoglobulin variable genes and design of family-specific oligonucleotide probes. Eur. J. Immunol. 21: 985-991 (1991)

### Example 4

### Exchange of an Expression Cassette Into Multiple High Expression Sites in an Expression Cell Line

The different insertion vectors each contain different positive selection markers (Blast, Hyg, Neo, Pur, etc.), so their integration into the genome can be selected. They also contain different homeostatic scorable markers (CJA8HA, CJA8 Flag, mCD4, mCD8, etc.), so the expression levels at each integration site can be measured. But these vectors share the same recombinase sites (FRT A, FRT B) and the same negative selection marker (HSV-TK), so that they can be exchanged simultaneously and cells which have not successfully exchanged all of the insertion cassettes can be selected against with acyclovir.

The method would involve transfecting the first vector, CE5, selecting integrants and choosing the highest expression clone based on its homeostatic scorable marker gene, CJA8Flag. This clone would then be transfected with the second integration vector, CE6, and repeating the clone selection process based on the second selectable marker and homeostatic scorable marker. This process could be repeated for a number of cycles until the desired number of high level expression sites had been modified with recombination cassettes. At this point the desired target gene could be introduced on an exchange vector carrying the same two recombination sites, FRT A and FRT B, flanking the target gene and a selectable marker, DHFR, along with a Flp recombinase expression cassette, CE9. Cells that had undergone successful exchange could be selected in methotrexate. Clones that had successfully exchanged all of the integration cassettes could be screened as CD4-+CD8-+CJA8Flag-+CJA8HA- or/and acyclovir resistant. The choice of the amplifiable marker gene on CE9, namely DHFR, would allow for positive selection of integrants in CHO dhfr-cells using methotrexate and could also allow further amplification of the target gene following the exchange event selecting with higher concentrations of methotrexate. This arrangement is preferred, but other positive selection markers could be used in CE9.

Although the invention has been described with reference to the presently preferred embodiments, it should be understood that various modifications can be made without departing from the spirit of the invention. All publications, patents, and patent applications are herein incorporated by reference in their entirety to the same extent as if each individual patent, or patent application was specifically and individually indicated to be incorporated by reference in its entirety.

What is claimed is:

1. A cellular expression system capable of exchanging at least one target gene, comprising:
a. a first integration cassette which comprises
i. a first promoter operably linked to
ii. a first exchangeable reporter segment having a first scorable homeostatic reporter element, which comprises at least one scorable reporter gene and an exchangeable reporter gene, the first scorable homeostatic reporter element linked at its 5′ end to a first recombinase recognition site, and at its 3′end to a second recombinase recognition site;
wherein the first integration cassette is capable of stable and random insertion into one or more first discrete genomic positions in a host cell, thereby creating a recombinant cell population;
b. a first target cassette comprising a first exchangeable target segment having:
i. a third recombinase recognition site, capable of recognizing the first recombinase recognition site in the first integration cassette,
ii. a first target element;
iii. a fourth recombinase recognition site, capable of recognizing the second recombinase recognition site in the first integration cassette;
wherein the first target element is linked at its 5′ end to the third recombinase recognition site, and at its 3′end to the fourth recombinase recognition site; and
c. at least one rec element encoding at least one recombinase activity recognizing the recombinase recognition sites of a and b,
wherein introduction of the rec element and the first target cassette to the recombinant cell population results in site-specific substitution of the first exchangeable reporter segment with the first exchangeable target segment at the first discrete genomic position.

2. The cellular expression system of claim 1, wherein the rec element is included in the first integration cassette.

3. The cellular expression system of claim 1, wherein the rec element is included in the first target cassette.

4. The cellular expression system of claim 1, wherein the recombinase activity is selected from the group consisting of Flp recombinase, Cre recombinase, Int recombinase, Sin recombinase and Hin recombinase.

5. The cellular expression system of claim 1, wherein the host cell is selected from the group consisting of mammalian cells, yeast cells and bacterial cells.

6. The cellular expression system of claim 1, wherein the first integration cassette further comprises a polycistronic element.

7. The cellular expression system of claim 1, wherein the first integration cassette further comprises a TAG sequence.

8. The cellular expression of claim 1, wherein the first target element further comprises a first target gene and a first selectable marker gene.

9. The cellular expression system of claim 8, wherein the first target cassette further comprises a polycistronic element.

10. The cellular expression system of claim 1, wherein the first target cassette further comprises a TAG sequence.

11. The cellular expression system of claim 1 further comprising:
d. a second integration cassette which comprises
i. a second promoter operably linked to
ii. a second exchangeable reporter segment having a second scorable homeostatic reporter element, which comprises at least one scorable reporter gene and an exchangeable reporter gene, the second scorable homeostatic reporter element linked at its 5′ end to a fifth recombinase recognition site, and at its 3′end to a sixth recombinase recognition site;
wherein the second integration cassette is capable of stable and random insertion into one or more second discrete genomic positions in a mammalian cell; and
e. a second target cassette comprising a second exchangeable target segment having:
i. a seventh recombinase recognition site, capable of recognizing the fifth recombinase recognition site in the second integration cassette;
ii. a second target element;
iii. an eighth recombinase recognition site, capable of recognizing the sixth recombinase recognition site in the second integration cassette;
wherein the second target element is linked at its 5′ end to the seventh recombinase recognition site, and at its 3′end to the eighth recombinase recognition site; and
f. a recombinase activity capable of recognizing the recombinase recognition sites of d and e;
wherein introduction of the second target cassette to the recombinant cell population results in site-specific substitution of the second exchangeable reporter segment with the second exchangeable target segment at the second discrete genomic position.

12. The cellular expression system of claim 1 1, wherein the second integration cassette further comprises a TAG sequence.

13. The cellular expression system of claim 1 1, wherein the second integration cassette further comprises a polycistronic element.

14. The cellular expression system of claim 11, wherein the second target element further comprises a second target gene and a selectable marker.

15. The cellular expression system of claim 14, wherein the second target cassette further comprises a polycistronic element.

16. The cellular expression system of claim 11, wherein the second target cassette further comprises a TAG sequence.

17. The cellular expression system of claim 11, wherein the first and second target elements each encode one subunit of a protein complex.

18. The cellular expression system of claim 17, wherein the protein complex is an antibody.

19. The cellular expression system of claim 11, wherein the first and second target elements encode one or more cloning sites.

20. An antibody library comprising:
a cell population, each cell of the cell population having a first integration cassette and a second integration cassette stably integrated at discrete genomic positions;
the first integration cassette comprising a promoter operably linked to a first nucleic acid encoding a first peptide for an antibody, the first nucleic acid linked at its 5′ end to a first recombinase recognition site, and at its 3′end to a second recombinase recognition site; and
the second integration cassette comprising a promoter operably linked to a second nucleic acid encoding a second peptide for an antibody, the second nucleic acid linked at its 5′ end to a third recombinase recognition site and at its 3′end to a fourth recombinase recognition site;
whereby the first and second nucleic acids are expressed at equal levels in each cell of the cell population.

21. The antibody library of claim 20, wherein the first nucleic acid comprises variable sequences.

22. The antibody library of claim 20, wherein the second nucleic acid comprises variable sequences.

23. The antibody library of claim 20, wherein the first peptide is an antibody light chain peptide and the second peptide is an antibody heavy chain peptide.

24. The antibody library of claim 20, wherein the first and second peptides are Fab peptides.

25. The antibody library of claim 20, wherein the first and second peptides are Fab′ peptides.

26. The antibody library of claim 20, wherein the first and second nucleic acids encode a humanized antibody peptide.

27. An integration cassette comprising:
a. a promoter operably linked to
b. an exchangeable reporter segment having a scorable homeostatic reporter element, which comprises at least one scorable reporter gene and an exchangeable reporter gene, the first scorable homeostatic reporter element linked at its 5′end to a first recombinase recognition site, and at its 3′end to a second recombinase recognition site;
wherein the integration cassette is capable of stable and random insertion into one or more discrete genomic positions in a host cell.

28. The integration cassette of claim 27 further comprising a TAG sequence.

29. The integration cassette of claim 27 further comprising a polycistronic element.

30. A method for selecting a transformed cell population capable of exchanging nucleic acid segments, comprising:
a. obtaining a first integration cassette as in claim 1(a);
b. introducing the first integration cassette into cells, creating a recombinant cell population with the first integration cassette stably inserted at one or more first discrete genomic positions within each cell;
c. scoring the level of expression of the first scorable homeostatic reporter element; and
d. selecting from the recombinant cell population those cells scoring a first predetermined level of expression for the first scorable homeostatic reporter element.

31. The method of claim 30, further comprising:
e. introducing to the selected recombinant cell population
i. a first target cassette as in claim 1(b);
ii. a rec element encoding recombinase activity recognizing the recombinase recognition sites of the first integration cassette and the first target cassette;
whereby the first exchangeable target segment is substituted for the first exchangeable reporter segment at the first discrete genomic positions.

32. The method of claim 31, wherein the recombinase activity of step (e) is chosen from the group consisting of Flp recombinase, Cre recombinase, Int recombinase, Sin recombinase and Hin recombinase.

33. The method of claim 30, wherein the first discrete genomic positions of step (b) are chromosomal.

34. The method of claim 30, wherein the first discrete genomic positions of step (b) are extrachromosomal.

35. The method of claim 30, wherein the scorable reporter gene encodes a surface antigen.

36. The method of claim 31, wherein the first target element further comprises a first target gene and a first selectable marker gene.

37. The method of claim 36, wherein substitution of the first exchangeable target segment for the first exchangeable reporter segment is monitored by screening for the absence of the scorable reporter gene and the presence of the first selectable marker gene.

38. The method of claim 30, wherein step d further comprises isolating a single cell from the population of cells scoring a first predetermined level of expression for the first scorable homeostatic reporter element, and
the method further comprising:
e. expanding the single cell to form a clonal cell population, wherein the first integration cassette is stably inserted at the same first discrete genomic positions within each cell of the clonal cell population.

39. The method of claim 31, wherein the first target element of step (e) has a secretory signal element.

40. The method of claim 31, further comprising:
f. obtaining a second integration cassette as in claim 11(d);
g. introducing the second integration cassette into the recombinant cell population of claim 30, thereby creating a second recombinant cell population with the second integration cassette inserted randomly at one or more second discrete genomic positions within each cell of the second recombinant cell population;
h. scoring the level of expression of the second scorable homeostatic reporter element for each cell of the second recombinant cell population; and
i. selecting from the second recombinant cell population those cells scoring a second predetermined level of expression for the second scorable homeostatic reporter element;
wherein the selected cells comprise the second integration cassette stably integrated at one or more second discrete genomic positions and the first integration cassette stably inserted at one or more first discrete genomic positions within each cell.

41. The method of claim 40, wherein the first scorable homeostatic reporter element and the second scorable homeostatic reporter element are expressed at equivalent levels.

42. The method of claim 40, wherein the first scorable homeostatic reporter element and the second scorable homeostatic reporter element are expressed at a preselected ratio.

43. The method of claim 40, wherein the second integration cassette further comprises a polycistronic element.

44. The method of claim 40, wherein the second integration cassette further comprises a TAG sequence.

45. The method of claim 40, wherein the second scorable homeostatic reporter element comprises a scorable reporter gene and an exchangeable reporter gene that differs from the first scorable homeostatic reporter element.

46. The method of claim 40, further comprising:
introducing to the second recombinant cell population;
i. a first target cassette as in claim 1 (b);
ii. a second target cassette as in claim 11 (e);
iii. a rec element encoding recombinase activity recognizing the recombinase recognition sites of the first and second integration cassettes and first and second target cassettes;
wherein the first exchangeable target segment is substituted for the first exchangeable reporter segment at the first discrete genomic positions, and the second exchangeable target segment is substituted for the second exchangeable reporter segment at the second discrete genomic positions.

47. The method of claim 46, wherein the first target cassette and the second target cassette encode subunits of a multi-subunit complex.

48. The method of claim 47, wherein the multi-subunit complex is an enzyme.

49. The method of claim 47, wherein the multi-subunit complex is an antibody.

50. A site-specific expression system comprising a recombinant cell population having an integration cassette as in claim 1(a), wherein the integration cassette is stably and randomly inserted at one or more discrete genomic positions within each cell of the recombinant cell population and wherein the homeostatic reporter element and the target element is expressed.

51. An antibody producing recombinant cell population, each cell of the recombinant cell population having a first integration cassette as in claim 1(a) and a second integration cassette as in claim 11(e), wherein each integration cassette is stably and randomly inserted at a first and second discrete genomic position, respectively, in each cell of the recombinant cell population, and wherein the first and second integration cassette is substituted with a first exchangeable target segment as in claim 1(b) and a second exchangeable target segment as in claim 11(f), wherein the first and second exchangeable target segment encodes an antibody chain, whereby the antibody chains encoded by the first and second exchangeable target segment is expressed at equivalent levels in each cell of the recombinant cell population.

52. The antibody producing recombinant cell population of claim 51, wherein the recombinant cell population is clonal in origin.

53. The antibody producing recombinant cell population of claim 51, wherein the antibody chains comprise a light chain and a heavy chain.

54. The antibody producing recombinant cell population of claim 53, wherein the heavy chain corresponds to a heavy chain Fab fragment.

55. The antibody producing recombinant cell population of claim 53, wherein the heavy chain corresponds to a heavy chain Fab′ fragment.

56. A recombinant expression cell line comprising:
a recombinant cell line having an integration cassette as in claim 1(a), wherein the integration cassette is stably inserted at a discrete genomic position that is identical in each cell of the recombinant cell line.

57. The recombinant expression cell line of claim 56, wherein the integration cassette further comprises a polycistronic element.

58. The recombinant expression cell line of claim 56, wherein the integration cassette further comprises a TAG sequence.

59. A method of making an antibody library comprising:
a. obtaining a second recombinant cell population as in claim 40(g);
wherein the first scorable homeostatic reporter element and the second scorable homeostatic reporter element are expressed at equivalent levels in the second recombinant cell population;
b. introducing a first target cassette having a first target element as in claim 1(b), wherein the first target element encodes a first peptide for an antibody; and
c. introducing a second target cassette having a second target element as in claim 11(e), wherein the second target element encodes a second peptide for an antibody;
whereby the first and second target elements are expressed at equal levels in each cell of the cell population.

60. The method of claim 59, wherein the first peptide comprises variable sequences.

61. The method of claim 59, wherein the second peptide comprises variable sequences.

62. The method of claim 59, wherein the first peptide is an antibody light chain peptide and the second peptide is an antibody heavy chain peptide.

63. The method of claim 59, wherein the first and second peptides are Fab peptides.

64. The method of claim 59, wherein the first and second peptides are Fab′ peptides.

65. The method of claim 59, wherein the first and second peptides are humanized antibody peptides.
